(19) United States
(12) Patent Application Publication
(10) Pub. No.: US 2005/0153978 A1
(43) Pub. Date: Jul. 14, 2005

(54) Medicaments

(76) Inventors: Michael John Alberti, Durham, NC (US); Stephen Garland, Harlow (GB); David Kendall Jung, Durham, NC (US); James Marvin Veal, Apex, NC (US); Jason Witherington, Harlow (GB)

Correspondence Address: SMITHKLINE BEECHAM CORPORATION;CORPORATE INTELLECTUAL PROPERTY-US, UW2220, P. O. BOX 1539, KING OF PRUSSIA, PA, 19406-0939, US

(21) Appl. No.: 10/508,592

(22) PCT Filed: Mar. 21, 2003
(86) PCT No.: PCT/GB03/01210

(30) Foreign Application Priority Data
Mar. 22, 2002 (GB) 0206861.7

(51) Int. Cl. A61K 31/496; A61K 31/4709; A61K 31/4439; A61K 31/4184; C07D 43/14

(52) U.S. Cl. 514/254.06; 514/310; 514/338; 514/394; 544/368; 546/146; 546/273.4; 548/304.7

(57) ABSTRACT

A method of treating an Msk-1 and/or ROCK(1 and 2) mediated disease or condition in a mammal comprising administration of an effective amount of a compounds of the formula (I) 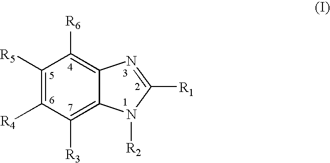 and physiologically acceptable salts thereof wherein,
R₁ is a 5, or 6 membered heterocyclic group selected from group a, b, c or d 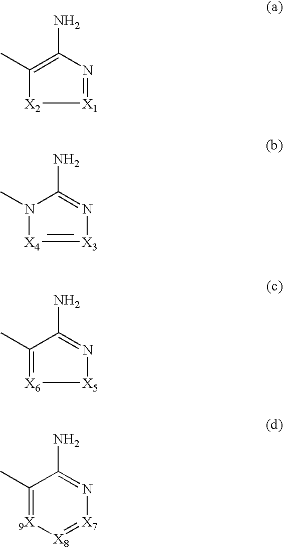
   wherein X₁ is a group selected from N or CR₇ and X₂ is a group selected from O, S or NR₈; X₃ and X₄ which may be the same or different is a group selected from N or CR₇; X₅ is a group selected from O, S or NR₈ and X₆ is N or CR₇; X₇, X₈ and X₉ may be the same or different and selected from a group N or CR₇, pharmaceutical compositions, novel compounds and processes for their preparation.

The present invention relates to the use of kinase inhibitors in the treatment of disease, to pharmaceutical compositions for use therein, to novel compounds and to processes for their preparation. More particularly the present invention relates to the use of benzimidazole derivatives which are inhibitors of the kinases mitogen and stress activated protein kinase-1 (herein after referred to as Msk-1) and/or Rho-kinase (hereinafter referred to as ROCK 1 and 2) in the treatment or prophylaxis of diseases and/or conditions mediated through the kinases Msk-1 and/or ROCK 1 and/or 2.

An important mechanism by which cells sense and respond to extracellular stimuli is the activation and modulation of intracellular signal transduction pathways. One of the major signal transduction systems utilized by cells is the MAPK signalling pathways. These pathways share a common architecture, consisting of a cascade of protein kinases that are sequentially phosphorylated and activated, resulting in the activation of a MAP kinase (MAPK). Three MAP kinase pathways have been widely characterised: the Erk pathway, which responds to mitogenic stimuli and results in activation of Erk, and the JNK and p38 pathways, which are commonly associated with transducing cellular stress signals and result in activation of JNK and p38 MAPK.

Mitogen and stress-activated protein kinases 1 (Msk1) and 2 (Msk2, also named RSKB or RLPK) constitute a family of kinases that can be phosphorylated and activated by either p38 or Erk. Msks are reported to be localized exclusively to the nucleus, and are responsible for the phosphorylation and activation of the transcription factor CREB in response to certain stress stimuli. In macrophage and monocyte cells, Msk1 is involved in CREB-mediated transcriptional regulation of IL-1β and Cox2 in response to bacterial lipopolysaccharide. In addition, Msk1 can also phosphorylate the nucleosomal proteins histone H3 and HMG14, and thus may have a critical role in linking cellular signalling pathways to chromatin modification and modulation of transcription factor complexes. Inhibitors of kinases in the Erk MAPK cascade have been suggested for use in the treatment and/or prophylaxis of disorders associated with neuronal degeneration resulting from ischemic events, including cerebral ischemia after cardiac arrest, stroke and multi-infarct dementia and also after cerebral ischemic events such as those resulting from head injury, surgery and/or during childbirth. Since Msks are activated by Erk MAPK, Msk inhibitors could serve a similar use. Although Msks are only one of a number of Erk substrates, CREB is involved in many different transcriptional activities, and Msk-mediated CREB phosphorylation could play a role in some cancers. In addition, through modulation of production of pro-inflammatory cytokines such as IL-1β and prostaglandins, inhibitors of Msks could be of use in treatments for neuroinflammatory diseases such as stroke, multiple sclerosis, Alzheimers disease, Parkinson's disease, amyotrophic lateral sclerosis and inflammatory pain, as well as other inflammatory diseases such as rheumatoid arthritis, irritable bowel syndrome, inflammatory bowel disease and asthma.

Another of the major signal transduction systems utilized by cells is the RhoA-signalling pathways. RhoA is a small GTP binding protein that can be activated by several extracellular stimuli such as growth factor, hormones, mechanic stress, osmotic change as well as high concentration of metabolites like glucose. RhoA activation involves GTP binding, conformation alteration, post-translational modification (geranylgeranyllization and farnesylation) and activation of its intrinsic GTPase activity. Activated RhoA is capable of interacting with several effector proteins including ROCKs and transmit signals into cellular cytoplasm and nucleus.

ROCK1 and 2 constitute a family of kinases that can be activated by RhoA-GTP complex via physical association. Activated ROCKs phosphorylate a number of substrates and play important roles in pivotal cellular functions. The substrates for ROCKs include myosin binding subunit of myosin light chain phosphatase (MBS, also named MYPT1), adducin, moesin, myosin light chain (MLC), LIM kinase as well as transcription factor FHL. The phosphorylation of theses substrates modulate the biological activity of the proteins and thus provide a means to alter cell's response to external stimuli. One well documented example is the participation of ROCK in smooth muscle contraction. Upon stimulation by phenylephrine, smooth muscle from blood vessels contracts. Studies have shown that phenylephrine stimulates b-adrenergic receptors and leads to the activation of RhoA. Activated RhoA in turn stimulates kinase activity of ROCK1 and which in turn phosphorylates MBS. Such phosphorylation inhibits the enzyme activity of myosin light chain phosphatase and increases the phosphorylation of myosin light chain itself by a calcium-dependent myosin light chain kinase (MLCK) and consequently increases the contractility of myosin-actin bundle, leading to smooth muscle contraction. This phenomena is also sometimes called calcium sensitization. In addition to smooth muscle contraction, ROCKs have also been shown to be involved in cellular functions including apoptosis, cell migration, transcriptional activation, fibrosis, cytokinesis, inflammation and cell proliferation. Moreover, in neurons ROCK plays a critical role in the inhibition of axonal growth by myelin-associated inhibitory factors such as myelin-associated glycoprotein (MAG). ROCK-activity also mediates the collapse of growth cones in developing neurons. Both processes are thought to be mediated by ROCK-induced phosphorylation of substrates such as LIM kinase and myosin light chain phosphatase, resulting in increased contractility of the neuronal actin-myosin system.

Inhibitors of ROCKs have been suggested for use in the treatments of a variety of diseases. They include cardiovascular diseases such as hypertension, chronic and congestive heart failure, cardiac hypertrophy, restenosis, chronic renal failure and atherosclerosis. In addition, because of its muscle relaxing properties, it is also suitable for asthma, female sexual dysfunction, male erectile dysfunctions and over-active bladder syndrome. ROCK inhibitors have been shown to possess anti-inflammatory properties. Thus they can be used as treatment for neuroinflammatory diseases such as stroke, multiple sclerosis, Alzheimer's disease, Parkinson's disease, amyotrophic lateral sclerosis and inflammatory pain, as well as other inflammatory diseases such as rheumatoid arthritis, irritable bowel syndrome, inflammatory bowel disease. In addition, based on their neurite outgrowth inducing effects, ROCK inhibitors could be useful drugs for neuronal regeneration, inducing new axonal growth and axonal rewiring across lesions within the CNS. ROCK inhibitors are therefore likely to be useful for regenerative (recovery) treatment of CNS disorders such as spinal cord injury, acute neuronal injury (stroke, traumatic brain injury), Parkinsons disease, Alzheimers disease and other neurodegenerative disorders. Since ROCK inhibitors reduce cell proliferation and cell migration, they could be useful in treating cancer and tumor metastasis. Furthermore, there is evidence suggesting that ROCK inhibitors suppress cytoskeletal rearrangement upon virus invasion, thus they also have potential therapeutic value in anti-viral and anti-bacterial applications. ROCK inhibitors are also useful for the treatment of insulin resistance and diabetes.

WO 97/12615 teaches inter alia novel 2-heteroaryl benzimidazole derivatives, which are inhibitors of the specific lipoxygenase enzyme 15-LO.

We have now identified a group of benzimidazole derivatives which are potent inhibitors of the protein kinases Msk-1 and/or ROCK(1 and/or 2).

The present invention thus provides a method of treating an Msk-1 and/or ROCK(1 and 2) mediated disease or condition in a mammal comprising administration of an effective amount of a compounds of the general formula (I) 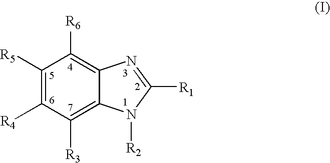
and physiologically acceptable salts thereof wherein,
R₁ is a 5, or 6 membered heterocyclic group selected from group a, b, c or d 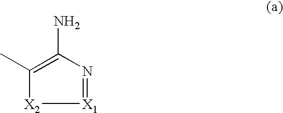
   wherein X₁ is a group selected from N or CR₇ and X₂ is a group selected from O, S or 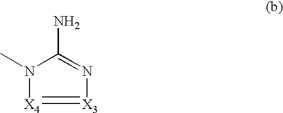
   wherein X₃ and X₄ which may be the same or different is a group selected from N or CR₇; 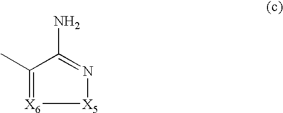
   wherein X₅ is a group selected from O, S or NR₈ and X₆ is N or CR₇; 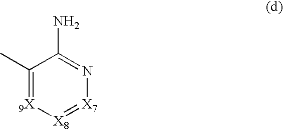
   wherein X₇, X₈ and X₉ may be the same or different and selected from a group N or CR₇ R₂ and R₈ independently represents hydrogen, hydroxy, aryl, heteroaryl, C₃₋₇cycloalkyl, heterocyclyl, a group YR₉, N═R₁₀, CONR₁₁R₁₂, COCH₂NR₁₁R₁₂,NR₁₁ COR₁₃, SO₂NR₁₁R₁₂ or C₁₋₆alkyl [optionally substituted by a group selected from optionally substituted phenyl, C₃₋₇cycloalkyl, heteroaryl, heterocyclyl, acylamino, NH₂, R₁₆NH, R₁₆R₁₇N, SO₂NR₁₁R₁₂, CONR₁₁R₁₂, NHCOR₁₃, OalkNR₁₆R₁₇, SalkNR₁₆R₁₇ or NR₁₄SO₂R₁₅ group]; or R₂ and R₃ together form a C₂₋₄ alkylene chain.
R₃, R₄, R₅, R₆ and R₇ independently represent a group selected from hydrogen, halogen, hydroxy, R₁₆O, R₁₆S(O)ₙ, NH₂, R₁₆NH, R₁₆R₁₇N, nitro, formyl, C₁₋₄alkanoyl, alkenyl (optionally substituted by optionally substituted phenyl, heterocyclyl, or heteoaryl), carboxy, optionally substituted phenyl, heteroaryl, cycloalkyl, cycloalkylalkyl, aryloxy, heteroaryloxy, heterocyclyl, CONR₁₁R₁₂, NR₁₁ COR₁₃, SO₂NR₁₁R₁₂, NR₁₄SO₂R₅ or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, acylamino, hydroxy, CONR₁₁R₁₂, NR₁₁COR₁₃, SO₂NR₁₁R₁₂, NR₁₄SO₂R₁₅. OalkNR₁₆R₁₇, or SalkNR₁₆R₁₇ group];
Y represents O, NH,NR₉ or S(O)ₙ;
R₉ represents aryl, heteroaryl, cycloalkyl, heterocyclyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, acylamino, hydroxy, CONR₁₁R₁₂, NR₁₁COR₁₃, SO₂NR₁₁, R₁₂, NR₁₄SO₂R₁₅, OalkNR₁₆R₁₇, or SalkNR₁₆R₁₇ group];
   R₁₀ represents an alkylidene group which may be substituted by an aryl, heteroaryl, heterocyclyl or cycloalkyl group or R₁₀ represents a cycloalkylidene or heterocycloalkylidene group.
   R₁₁ and R₁₂ independently represent hydrogen, aryl, heteroaryl, cycloalkyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, or acylamino group] or R₁₁ and R₁₂ together with the nitrogen atom to which they are attached form a 4-7 heterocyclic ring which may be saturated or unsaturated and optionally contains another heteroatom selected from O,N or S(O)ₙ;
   R13 and R₁₅ independently represent, aryl, heteroaryl, heterocyclyl, cycloalkyl or C₁₋₆alkyl [optionally substituted by a group selected from optionally substituted phenyl, C₃₋₇cloalkyl, heteroaryl, heterocyclyl, NH₂, R₁₆NH, R₁₆R₁₇N, or acylamino group] or the group NR₁₁R₁₂ wherein R₁₁ and R₁ have the meanings defined above;
   R₁₄ represents hydrogen, aryl, heteroaryl, heterocyclyl, cycloalkyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, or acylamino group];
R₁₆ and R₁₇ independently represent a group selected from C₁₋₆alkyl, C₃₋₇cycloalkyl, C₃₋₇cycloalkylalkyl, aryl, aralkyl, heteroaryl, heteroarylalkyl, heterocyclyl or heterocyclylalkyl;
   Alk is a C₂₋₄ straight or branched alkylene chain
n is zero, 1 or 2.

It will be appreciated that any of the substituents R₁ to R₁₇ as defined in formula (1) above may contain at least one asymmetric center and it is to be understood that the invention includes all possible enantiomers arising there from and mixtures thereof including racemates.

The term alkyl as a group or part of a group e.g. alkoxy, alkylthio, alkylamino, dialkylamino, optionally substituted alkyl e.g. aminoalkyl, cycloalkylalkyl, aralkyl, heteroarylalkyl or heterocyclylalkyl refers to a C₁₋₆ straight or branched chain alkyl group.

The term halogen includes fluorine, chlorine, bromine or iodine.

The term aryl as a group or part of a group e.g. aryloxy, aralkyl or arylamino refers to an optionally substituted phenyl or fused bicyclic aryl group e.g. naphthyl.

The terms aryl, optionally substituted phenyl, heteroaryl, C₃₋₇ cycloalkyl as a group or part of a group and 4-7 membered heterocyclyl as a group or part of a group includes such groups which are optionally substituted with 1 to 3 substituents which may be the same or different and selected from halogen, aryl, heteroaryl, heterocyclylalkyl, hydroxy, alkyl, alkoxy, trifluoroalkyl, amino, alkylamino, dialkylamino, arylamino, heteroarylamino, heterocyclylamino, acylamino, aminoalkyl, alkylaminoalkyl, dialkylaminoalkyl, acylaminoalkyl, arylaminoalkyl, heteroarylaminoalkyl, cycloalkylaminoalkyl, heteroclylaminoalkyl, hydroxyalkyl, CONR₁₁R₁₂, CH₂CONR₁₁R₁₂ carboxy, carboxamido, alkoxycarbonyl, aminoalkoxy, dialkylaminoalkoxy, acylaminoalkoxy, sulphonamido, aminosulphonyl, cyano, formyl, nitro, R₁₈O or R₁₈S(O)ₙ wherein R₁₈ is a group selected from alkyl, aryl, heteroaryl or heterocyclylalkoxy and n is zero, one or two, or each of the said groups form part of a fused bicyclic ring system containing up to 10 ring members and which is at least partially saturated.

The term heteroaryl as a group or part of a group e.g. heteroaryloxy refers to a 5 or 6 membered ring or a fused 5, 6 or 6,6 bicyclic ring system.

When heteroaryl represents a 5 membered group it contains a heteroatom selected from O, N or S and may optionally contain a further 1 to 3 nitrogen atoms. Examples of such groups include furanyl, thienyl, isoxazolyl, oxazolyl or imidazolyl. When heteroaryl represents a 6-membered group it contains from 1 to 3 nitrogen atoms. Examples of such groups include pyridyl, pyrimidinyl, or triazinyl.

The term 5,6 fused bicyclic heteroaryl group refers to a group in which the 5-membered ring contains an oxygen, sulphur or NH group and the 6 membered ring optionally contains from 1 to 3 nitrogen atoms. Examples of such groups include benzofuranyl, benzothienyl or indolyl.

The term 6,6-fused bicyclic heteroaryl group refers to a bicyclic heteroaryl group which contains at least one nitrogen atom in one of the rings and may contain up to 3 nitrogen atoms in each ring. Examples of such groups include quinolinyl, isoquinolinyl or naphthyridinyl also the term 6,6 fused bicyclic heteroaryl group refers to a 6-membered heteroaryl group which is fused to a partially saturated carbocyclic group. Examples of such a group includes tetrahydroquinolinyl or tetrahydroisoquinolinyl.

The term heterocyclyl as a group or part of a group e.g. heterocyclylalkyl or heterocyclylalkylidene refers to a 4-7 membered heterocyclyl group which is linked to the rest of the compound of formula (1) via a carbon or nitrogen atom in that group and which contains one or two hetero atoms selected from N, O or S(O)ₙ, and when the heterocyclyl group contains a ring member NH or the heterocyclyl group is substituted by a primary or secondary amino group then the term also includes N-alkyl, N-optionally substituted phenyl, N-benzyl or, N-acyl derivatives thereof. Examples of such heterocyclic groups include optionally substituted pyrrolidine, piperidine, piperazine homopiperazine, morpholine or thiomorpholine.

The term cycloalkyl as a group or part of a group e.g. cycloalkylalkyl or cycloalkylidene refers to a 3-7 membered carbocyclic group.

The term fused bicyclic ring system containing up to 10 ring members and which is at least partially saturated includes carbocyclic and heterocyclic 6,5 and 6,6 bicyclic ring systems. Examples of such 6,5 and 6,6 carbocyclic ring systems include those wherein the bicyclic ring comprises a benzene ring fused to a 5- or 6-membered carbocyclic ring which is at least partially saturated e.g. tetrahydronaphthyl, indanyl or indenyl. Examples of such 6,5, or 6,6 heterocyclic rings include those wherein one ring is benzene which is fused to a 5 or 6 membered ring containing one or two hetero atoms selected from O, S or N e.g. indolinyl, isoindolinyl, 2,3-dihydro-1H-isoindol-5-yl, dihydrobenzofuranyl, dihydrobenzothienyl, 1,3-benzodioxolyl, benzopyrrolyl, 1,3-benozodithiolyl, 1,4-benzodioxanyl, chromanyl or chromenyl.

The term acyl as a group or part of the acylamino group referes to an alkanoyl, aroyl, aralkanoyl, alkoxycarbonyl, aryloxycarbonyl or aralkoxycarbonyl group.

The compounds of formula (I) form salt with inorganic and organic acids and the invention includes such salts formed with physiologically acceptable inorganic and organic acids.

A preferred class of compounds of formula (I) are those wherein R₁ is a group (c). When R₁ is the group (c) this is conveniently a group wherein X₅ is oxygen and X₆ is nitrogen or X₅ is NR₈ wherein R₈ is hydrogen or methyl and X₆ is CH.

The group R₁ is preferably a group (c) wherein X₅ is oxygen and X₆ is nitrogen. Examples of suitable R₂ groups include hydrogen C₁₋₆alkyl such as methyl, ethyl, propyl, isopropyl, butyl, alkenyl e.g. allyl, alkynyl e.g. propargyl, C₃₋₇ cycloalkyl e.g. cyclopropyl or cyclohexyl, C₃₋₇cycloakylalkyl e.g. C₃₋₇cycloalkylmethyl such as cyclopropylmethyl or cyclobutylmethyl, optionally substituted phenyl such as phenyl or phenyl (substituted by halogen e.g. chlorine, or aminomethyl), heteroaryl such as pyridyl e.g. 3-pyridyl, or triazinyl e.g, 2,4-diamino-1,3,5-triazinyl, alkyl (substituted by amino, R₁₆NH or R₁₆R₁₇N such as 2-aminoethyl, 2-dimethylaminoethyl, 2-amino-1-methylethyl, 2-dimethylamino-1-methylethyl), or 4-7 membered heterocyclyl group (such as 4-piperidinyl, t-butyloxycarbonyl-piperidinyl, alkyl [substituted by a 4-7 membered heterocyclyl group e.g 1-ethylpyrrolidin-2-yl-methyl, 3-(4-methyl piperazinyl-1-yl)propyl)], a 6 membered heteroaryl group fused to a partially saturated carbocyclic ring e.g 1,2,3,4 tetrahydroisoquinolin-7-yl and the N-t-butoxycarbonyl derivative thereof, alkyl substituted by optionally substituted phenyl e.g. 1-phenylethyl, alkyl substituted by alkenyloxy e.g. 2-vinyloxyethyl, or alkyl substituted by trifluoromethyl e.g. 2,2,2-trifluoroethyl.

Conveniently the group R₃ is hydrogen or R₃ and R₂ together represent propylene chain.

The group R₄ is conveniently hydrogen, halogen e.g. chlorine or fluorine, dimethylamino, phenoxy or hydroxy.

The group R₅ is conveniently hydrogen, alkyl e.g. methyl, halogen e.g. bromine, or chlorine, alkoxy e.g. ethoxy, formyl, acetyl, hydroxymethyl or hydroxy.

R₆ is convenientely hydrogen, ethylamino or nitro.

Specific preferred compounds according to the invention include:
4-(1-Ethyl-1H-benzoimidazol-2-yl)-furazan-3-ylamine;
4-(1H-Benzoimidazol-2-yl)-furazan-3-ylamine;
4-[2-(Amino-furazan-3-yl)-benzoimidazol-1-yl]-piperidine-1 arboxylic acid tert-butyl ester;
4-(1-Piperidin-4-yl-1H-benzoimidazol-2-yl)-furazan-3-ylamine;
4-[1-(2-Amino-ethyl)-1H-benzoimidazol-2-yl]-furazan-3-ylamine;
4-[1-(2-Amino-1-methylethyl)-1-H-benzoimidazol-2-yl]furazan-3-ylamine;
4-(5-Bromo-1-ethyl-1H-benzoimidazol-2-yl)furazan-3-ylamine;
4-(1-Cyclopropyl-1H-benzoimidazol-2-yl)-furazan-3-ylamine;
4-(1-Cyclohexyl-1H-benzoimidazol-2-yl)-furazan-3-ylamine;
4-(1-Cyclopropylmethyl-1H-benzoimidazol-2-yl)-furazan-3-ylamine;
4-[1-(2-Dimethylamino-ethyl)-1H-benzoimidazol-2-yl]-furazan-3-ylamine;
4-[1-(2-Dimethylamino-1-methyl-ethyl)-1H-benzoimidazol-2-yl]-furazan-3-ylamine;
4-[1-(3-Chlorophenyl)-1-H-benzoimidazol-2-yl]furazan-3-ylamine;
4-[1-(4-Chlorophenyl)-1H-benzoimidazol-2-yl]furazan-3-ylamine;
4-(6-Chloro-1-ethyl-1H-benzoimidazol-2-yl)furazan-3-ylamine;
4-(1-Ethyl-5-methoxy-1H-benzoimidazol-2-yl)-furazan-3-ylamine;
4-(4-Nitro-1H-benzimidazol-2-yl)-furazan-3-ylamine;
4-(5,6-dihydro-4H-imidazo[4,5,1-ij]quinolin-2-yl)-furazan-3-ylamine;
4-(1-phenyl-1H-benzimidazol-2-yl)-furazan-3-ylamine;
4-(5-chloro-6-fluoro-1H-benzimidazol-2-yl)-furazan-3-ylamine;
4-(1-methyl-1H-benzimidazol-2-yl)-furazan-3-ylamine;
4-(6-fluoro-1H-benzimidazol-2-yl)-furazan-3-ylamine;
4-(1-pyridin-3-yl-1H-benzimidazol-2-yl)-furazan-3-ylamine;
[2-(4-Amino-furazan-3-yl)-1-ethyl-1H-benzoimidazol-5-yl]-ethanone;
2-(4-Amino-furazan-3-yl)-1-ethyl-1H-benzoimidazole-5-carbaldehyde;
[2-(4-Amino-furazan-3-yl)-ethyl-1H-benzoimidazol-5-yl]-methanol;
2-(4-Aminofurazan-3-yl)-3-ethyl-3H-benzoimidazol-5-ol;
2-(4-Aminofurazan-3-yl)-1-ethyl-1H-benzoimidazol-5-ol;
4-(1-Ethyl-4-nitro-1H-benzimidazol-2-yl)-furazan-3-ylamine;
4-(1-Ethyl-1H-benzoimidazol-2-yl)-1H-pyrazol-3-ylamine;
4-[1-(2,2,2-Trifluoroethyl)-1H-benzimidazol-2-yl]-furazan-3-ylamine;
4-{1-[3-(4-Methylpiperazin-1-yl)propyl]-1H-benzimidazol-2-yl}-furazan-3-ylamine; 4-{-[(1-Ethylpyrrolidin-2-yl)methyl]-1H-benzimidazol-2-yl}furazan-3-ylamine;
[2-(4-Amino-furazan-3-yl)-1-ethyl-1H-benzimidazol-4yl]-ethylamine;
4-(1-Propyl-1H-benzimidazol-2-yl)-furazan-3-ylamine;
4-[1-(Cyclobutylmethyl)-1H-benzimidazol-2-yl]-furazan-3-ylamine;
4-(1-Butyl-1H-benzimidazol-2-yl)-furazan-3-ylamine;
4-(1-Ethyl-5-fluoro-1H-benzimidazol-2-yl)-furazan-3-ylamine;
4-(1-Ethyl-6-fluoro-1H-benzimidazol-2-yl)-furazan-3-ylamine;
4-{1-[(1S)-1-Phenylethyl]-1H-benzimidazol-2-yl}furazan-3-ylamine.
4-[1-Ethyl-6-(methyloxy)-1H-benzimidazol-2-yl]-furazan-3-ylamine
2-(4-Amino-1,2,5-furazan-3-yl)-1-ethyl-N,N-dimethyl-1H-benzimidazol-6-amine
1,1-Dimethylethyl 7-[2-(4-amino-furazan-3-yl)-6-hydroxy-1H-benzimidazol-1-yl]-3,4 dihydro-2(1H)-isoquinolinecarboxylate
2-(4-Amino-furazan-3-yl)-1-(1,2,3,4-tetrahydro-7-isoquinolinyl)-1H-benzimidazol-6-ol
1,1-Dimethylethyl 7-[2-(4-amino-furazan-3-yl)-6-fluoro-1H-benzimidazol-1-yl]-3,4-dihydro-2(1H)-isoquinolinecarboxylate
4-[1-Ethyl-6-(phenyloxy)-1H-benzimidazol-2-yl]-furazan-3-ylamine

The ability of the compounds of formula (I) to antagonise the effect of the kinase Msk-1 may be determined using published procedures such as those described in WO9967283 and WO0127315. Alternatively the following in vitro assay may be used.

Thus the Msk-1 antagonist activity was determined using human recombinant Msk-1 expressed in Sf9 cells (WO9967283). The enzyme underwent prior activation by incubation with MAPK (p42), which was removed prior to storage and subsequent assay. The assay of Msk-1 activity involved incubation with peptide substrate and ATP³³, the subsequent incorporation of p33 into the peptide was quantified by Scintillation Proximity Assay (SPA-Amersham Pharmacia).

For IC50 determination, test compounds were typically dissolved at 10 mM in 100% DMSO, with subsequent serial dilution into 10% DMSO. Compounds were typically assayed over an eleven point dilution range with a concentration in the assay of 10 uM to 3 nM, in duplicate. IC50 values were calculated by bespoke curve fitting software.

Assays were performed in clear bottomed, white walled, 384 well plates, in a total assay volume of 12.5 ul. The assays contained: 2 nM activated MSK1; 2 uM biotinylated peptide (biotin-GRPRTSSFAEG-OH); 20 uM ATP; 25Bq per pmole ATP³³; 50 mM Hepes; 10 mM MgCl₂; 0.1 mM EDTA; 0.0025% Tween-20; 5 mM β-Mercaptoethanol; pH 7.5. The reactions were incubated at 20° C. for 60 minutes, then terminated by the addition of 10 ul of 200 mM EDTA.

Streptavidin PVT SPA beads were added to a concentration of 0.2 mg per well. The plates were shaken for 10 minutes before centrifugation at 2500 rpm for 10 minutes. P³³ incorporation was quantified by scintillation counting in a Wallac Trilux.

The compounds of the invention are therefore useful in the treatment or prevention of diseases and/or conditions mediated through the kinase Msk-1. Thus the compounds are useful for the treatment or prophylaxis of disorders associated with neuronal degeneration resulting from ischemic events or inflammatory conditions.

Examples of such disorders include acute stroke e.g. cerebral stroke, thromboembolic stroke, hemorrhagic stroke and cerebral ischemia, multi infarct dementia, pain, arthritis e.g. rheumatoid arthritis, osteoarthritic, psoriasis, and enteropathic arthritis, multiple sclerosis, Alzheimers disease, Parkinson's disease, amyotrophic lateral sclerosis, spinal cord injury and asthma. The compounds may also be useful for the treatment of irritable bowel syndrome, inflammatory bowel disease and certain cancers.

The ability of the compounds of formula (I) to antagonise the effect of the kinase ROCK1 may be determined by using the following assays:

1. ROCK Kinase Assay:

ROCK inhibitor activity was determined using human recombinant ROCK1 kinase domain (amino acid 1-578) expressed in Sf9 cells (WO9967283). The enzyme was purified using his-tag NTA column and Source 15 HPLC chromatography. The assay of Rock-1 activity involved incubation with peptide substrate and ATP³³, the subsequent incorporation of p33 into the peptide was quantified by Scintillation Proximity Assay (SPA-Amersham Pharmacia).

For IC50 determination, test compounds were typically dissolved at 10 mM in 100% DMSO, with subsequent serial dilution into 10% DMSO. Compounds were typically assayed over an eleven point dilution range with a concentration in the assay of 10 uM to 3 nM, in duplicate. IC50 values were calculated by bespoke curve fitting software.

Assays were performed in clear bottomed, white walled, 96 well plates, in a total assay volume of 40 ul. The assays contained: 1 nM hROCK1; 1 uM biotinylated peptide (biotin-Ahx-AKRRRLSSLRA-CONH2); 1 uM ATP; 25Bq per pmole ATP³³; 12.5 mM Hepes pH7.4; 7.5 mM MgCl₂; 0.015% BSA. The reactions were incubated at 20° C. for 120 minutes, then terminated by the addition of 10 ul of 200 mM EDTA.

Streptavidin PVT SPA beads were added to a concentration of 0.4 mg per well. The plates were shaken for 10 minutes before centrifugation at 2500 rpm for 10 minutes. P³³ incorporation was quantified by scintillation counting in a Wallac Trilux.

2. Aorta Artery Contraction Assay:

Male Sprague-Dawley rats (350-400 g) are anesthetized with 5% Isoflurane in O₂ and euthanized by exsanguination. Proximal descending thoracic aortae are removed and placed in oxygenated (95% O₂, 5% CO₂) ambient temperature Krebs solution (pH 7.4) with the following composition (mM): NaCl 112.0, KCl 4.7, CaCl₂ 2.5, KH₂PO₂ 1.2, MgSO₄ 1.2, NaHCO₃ 25.0, dextrose 11.0, indomethacin 0.01, and L-NAME 0.1. The isolated aortae are cut into four 3 mm rings and each segment is suspended by two 0.1 mm diameter tungsten wire hooks and placed in a 10 mL organ bath containing oxygenated (95% O₂, 5% CO₂) 37° C. Krebs solution (pH 7.4). The tissues are equilibrated under 1.0 grams resting tension for approximately 30 minutes. Responses are measured isometrically using a Grass FT 03 force-transducer and recorded on a Grass polygraph (model 7D) as change in tension.

After the equilibration period, each tissue is contracted with 60 mM KCl for about 15 minutes, washed with 37° C. Krebs solution, and allowed to relax to the resting tension. The 60 mM KCl contraction is repeated. The tissue is then contracted to equilibration with 1 □M norepinephrine and washed with 37° C. Krebs solution and allowed to relax to the resting tension.

A cumulative concentration-response curve to phenylephrine is obtained by dosing at 0.5 log unit intervals (1 nM to 1 □M) and the EC₈₀ is determined. Following several washes, each vessel is contracted to equilibrium with an EC₈₀ concentration of phenylephrine and tone is reversed by adding cumulative amounts of a ROCK inhibitor at 0.5 log unit intervals (0.1 nM to 3 □M). When constructing cumulative concentration-response, a higher concentration of vasoactive agent is added to the tissue bath after the previous response has reached a plateau.

3. In Vivo Effect of ROCK Inhibitors (Intravenous, IV):

Rats are anesthetised with isoflurane 3% for the implantation of femoral venous and arterial catheters. During the experiment, anaesthesia was maintained at 2%. Each rat received an intravenous bolus dose of vehicle followed by a bolus doses of ROCK inhibitors at 10, 30, 100 and 300 ug/kg with approx 20 minutes between doses. Blood samples are taken at 20 minutes after doses 10, 30 and 300 ug/kg for the subsequent determination of plasma levels of ROCK inhibitors.

Arterial blood pressure and heart rate derived from the blood pressure are recorded on strip chart and also on a computerised data acquisition system (Po—Ne-Mah, V 3.30, Gould Instruments). Hemodynamic data is presented as the maximum change from pre-dose control expressed as means +/−SEM.

The compounds of the invention are therefore useful in the treatment or prevention of diseases and/or conditions mediated through Rho kinases-1 and/or -2.

Thus the compounds are useful for the treatment or prophylaxis of cardiovascular diseases such as hypertension, chronic and congestive heart failure, cardiac hypertrophy, restenosis, chronic renal failure and atherosclerosis. The compounds are useful for the treatment or prophylaxis of disorders associated with neuroinflammatory diseases such as stroke, spinal cord injury, multiple sclerosis, Alzheimer's disease, Parkinson's disease, amyotrophic lateral sclerosis and/or inflammatory disorders such as inflammatory pain, as rheumatoid arthritis, irritable bowel syndrome, inflammatory bowel disease.

The compounds are useful for the treatment or prophylaxis of asthma, female sexual dysfunction, male erectile dysfunctions and over-active bladder syndrome, cancer and tumor metastasis, viral and bacterial infections, insulin resistance and diabetes.

The invention therefore provides for the use of a compound of formula (I) and/or physiologically acceptable salts thereof for use in therapy and in particular for use as a medicine for inhibiting the effects of the kinases Msk-1 and/or ROCK(1 and/or 2).

The invention also provides for the use of a compound of formula (I) and/or a physiologically acceptable derivative or salt thereof for the manufacture of a medicament for inhibiting the effects of the kinases Msk-1 and/or ROCK(1 and/or 2).

According to a further aspect, the invention also provides for a method for inhibiting the effects of the kinases Msk-1 and/or ROCK(1 and/or 2) in a mammal e.g. a human, comprising administering to a patient in need thereof an effective amount of a compound of formula (I) and/or a physiologically acceptable derivative or salt thereof.

It will be appreciated by those skilled in the art that reference herein to treatment extends to prophylaxis as well as the treatment of established diseases or symptoms.

It will further be appreciated that the amount of a compound of the invention required for use in treatment will vary with the nature of the condition being treated, the route of administration and the age and the condition of the patient and will be ultimately at the discretion of the attendant physician. In general however doses employed for adult human treatment will typically be in the range of 5 to 800 mg per day, dependent upon the route of administration.

Thus for parenteral administration a daily dose regimen will typically be in the range 0.1 to 80 mg/kg of the total body weight, preferably from about 0.2 to 30 mg/kg or more preferably 0.5 to 15 mg/kg. For oral administration a daily dose regimen will typically be within the range range 0.1 to 80 mg/kg of the total body weight, preferably from about 0.2 to 30 mg/kg or more preferably 0.5 to 15 mg/kg.

The desired dose may conveniently be presented in a single dose or as divided doses administered at appropriate intervals, for example as two, three, four or more sub-doses per day.

Preferred routes of administration include by intravenous injection or orally.

While it is possible that, for use in therapy, a compound of the invention may be administered as the raw chemical, it is preferable to present the active ingredient as a pharmaceutical formulation.

The invention thus further provides a pharmaceutical formulation comprising a compound of formula (I) or a pharmaceutically acceptable salt thereof together with one or more pharmaceutically acceptable carriers thereof and, optionally, other therapeutic and/or prophylactic ingredients. The carrier(s) must be ‘acceptable’ in the sense of being compatible with the other ingredients of the formulation and not deleterious to the recipient thereof.

The compositions of the invention include those in a form especially formulated for oral, buccal, parenteral, inhalation or insufflation, implant or rectal administration. Appropriate dosage forms for adminisration by each of these routes may be prepared by conventional techniques.

Tablets and capsules for oral administration may contain conventional excipients such as binding agents, for example, syrup, acacia, gelatin, sorbitol, tragacanth, mucilage of starch or polyvinylpyrrolidone; fillers, for example, lactose, sugar, microcystalline cellulose, maize-starch, calcium phosphate or sorbitol; lubricants, for example, magnesium stearate, stearic acid, talc, polyethylene glycol or silica; disintegrants, for example, potato starch or sodium starch glycollate, or wetting agents such as sodium lauryl sulphate. The tablets may be coated according to methods well known in the art. Oral liquid preparations may be in the form of, for example, aqueous or oily suspensions, solutions emulsions, syrups or elixirs, or may be presented as a dry product for constitution with water or other suitable vehicle before use. Such liquid preparations may contain conventional additives such as suspending agents, for example, sorbitol syrup, methyl cellulose, glucose/sugar syrup, gelatin, hydroxyethylcellulose, carboxymethyl cellulose, aluminium stearate gel or hydrogenated edible fats; emulsifying agents, for example, lecithin, sorbitan mono-oleate or acacia; non-aqueous vehicles (which may include edible oils), for example, almond oil, fractionated coconut oil, oily esters, propylene glycol or ethyl alcohol; solubilizers such as surfactants for example polysorbates or other agents such as cyclodextrins; and preservatives, for example, methyl or propyl p-hydroxybenzoates or ascorbic acid. The compositions may also be formulated as suppositories, e.g. containing conventional suppository bases such as cocoa butter or other glycerides.

For buccal administration the composition may take the form of tablets or lozenges formulated in conventional manner.

The composition according to the invention may be formulated for parenteral administration by injection or continuous infusion. Formulations for injection may be presented in unit dose form in ampoules, or in multi-dose containers with an added preservative. The compositions may take such forms as suspensions, solutions, or emulsions in oily or aqueous vehicles, and may contain formulatory agents such as suspending, stabilising and/or dispersing agents. Alternatively the active ingredient may be in powder form for constitution with a suitable vehicle, e.g. sterile, pyrogen-free water, before use.

Coveniently the compounds of the invention are formulated for intravenous or oral administration.

The compositions according to the invention may contain between 0.1-99% of the active ingredient, conveniently from 30-95% for tablets and capsules and 3-50% for liquid preparations.

The invention further provides novel compounds within the scope of the general formula (I), examples of such novel compounds include the compounds, the synthesis of which are specifically described in the examples.

Compounds of formula (I) wherein R₁ is a group (a), (c) and (d) may be prepared by reacting the diamine (II) 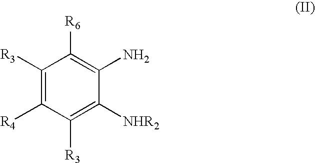
wherein R₂, R₃, R₄, R₅ and R₆ have the meanings defined in (I) with the appropriate compound of formula (III), (IV) or (V) 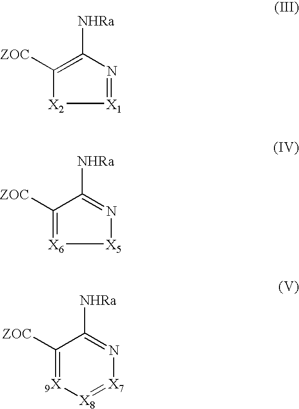
wherein Z is hydrogen, halogen e.g. Cl, Br or I, hydroxy or C₁₋₄alkoxy, Ra is hydrogen or a nitrogen protecting group such as an alkoxycarbonyloxy or benzyloxycarbonyloxy group and each of X₁, X₂, X₅, X₆, X₇, X₈ and X₉ have the meanings as defined in formula (I) or is a group available thereto, followed when required by removal of the nitrogen protecting group Ra using conventional methods.

When Z is a group selected from halogen, alkoxy or hydroxy the reaction is carried out with heating and optionally in the presence of a solvent and/or a dehydrating agent such as polyphosphoric acid.

When Z is hydrogen the reaction is conveniently carried out in the presence of an oxidant such as sodium bisulphite.

Compounds of formula (I) wherein R₁ is the group (b) may be prepared by reduction of the corresponding nitro derivative (VI) 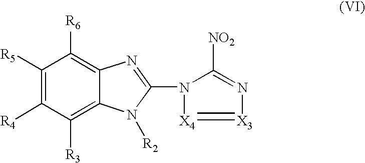
wherein R₂, R₃, R₄, R₅, R₆, X₃ and X₄ have the meanings defined in formula (I). The reduction may be effected using conventional procedures for converting a nitro group into an amino group, thus for example the reduction may be effected using hydrogen and a suitable metal catalyst e.g. palladium.

Compounds of formula (I) wherein R₁ is the group (c) and X₅ is oxygen and X₆ is nitrogen may be prepared by reacting the nitrile (VII) 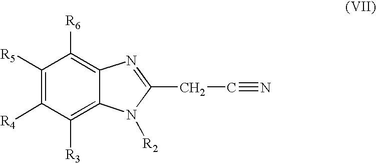
wherein R₂, R₃, R₄ R₅ and R₆ have the meanings defined in formula (I) with hydrochloric acid and sodium nitrite in a solvent such as an alkanol and treatment of the product thus formed with a base e.g. aqueous sodium hydroxide and hydroxylamine and subsequent heating.

Alternatively compounds of formula (I) wherein R₁ is the group (c) and X₅ is oxygen and X₆ is nitrogen may be prepared by reacting the diamine (II) with 5-methyl-[1,4,5]oxadiazolo[3,4-d]pyrimidin-7-ol in glacial acetic acid and with heating. In a modification of this process the reaction may be carried out using a derivative of the diamine(II) wherein the primary amino group is in a protected form e.g. as a t-butoxycarbonylamino group.

Compounds of formula (I) wherein R₁ represent the group (c) wherein X₅ is NH and X₆ is CH may be prepared by reacting compound (VIII) 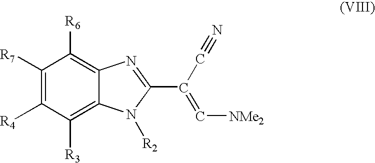
wherein R₂, R₃, R₄ R₅ and R₁ have the meanings defined in formula (I) with hydrazine. This reaction is preferably carried out in a solvent e.g. an alkanol such as methanol and with heating.

In another aspect of the invention compounds of formula (I) may be converted into other compounds of formula (I).

Thus compounds wherein R₂ is an optionally substituted alkyl group may be prepared by treating the corresponding compound wherein R₂ is hydrogen with the appropriate optionally substituted alkyl halide in a solvent such as DMF and in the presence of a base such as sodium hydride or cesium carbonate.

Compounds of formula (I) wherein R₅ is an acetyl group may be prepared by treating the corresponding compound of formula (I) wherein R₅ is halogen e.g. bromine with (1-ethoxyvinyl)tributyl tin and bis(triphenylphosphine) palladium (II) chloride in a solvent such as toluene. Conveniently the reaction is carried out with heating in an atmosphere of argon.

Compounds of formula (I) wherein R₅ is a formyl group may be prepared by treating the corresponding compound of formula (I) wherein R₅ is halogen e.g. bromine with bis(triphenylphosphine) palladium (II) chloride and sodium formate in a solvent such as DMF. Conveniently the reaction is carried out with heating in an atmosphere of carbon monoxide.

A compound of formula (I) wherein R₅ is hydroxymethyl may be prepared by reaction of the corresponding compound of formula (I) wherein R₅ is formyl using sodium borohydride in a solvent such as an alkanol e.g. anhydrous methanol.

The nitrile of formula (VII) may be prepared by heating of a compound of formula (II) with ethyl cyanoacetate.

The compound of formula (VIII) may be prepared by reacting the nitrile of formula (VII) with an acetal of N,N dimethylformamide in a solvent e.g. a hydrocarbon such as ortho xylene and with heating.

Compounds of formula (VI) may be prepared by reaction of the 2-iodo-benzimidazole derivative (IX) 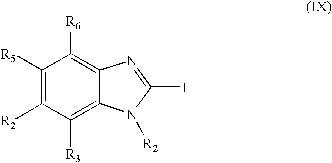
wherein R₂, R₃, R₄, R₅ and R₆ have the meanings defined in formula (I) with the compound (X) 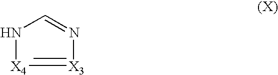
wherein X₃ and X₄ have the meanings defined in formula (I) in the presence of a base and a polar aprotic solvent and then reacting the resultant compound (XI). 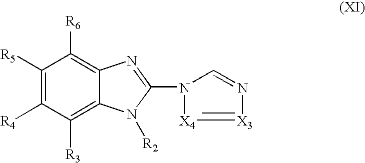
with an alkyl nitrite in the presence of a suitable base.

Compounds of formula (II) may be prepared by reacting a compound of formula (XII) 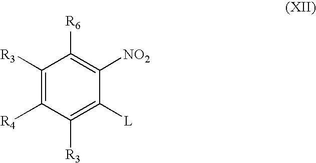
wherein R₃, R₄, R₅ and R₆ have the meanings defined above and L is a group displaceable by the amine R₂NH₂ e.g. methoxy, bromine, chlorine, fluorine or methoxysulphonyl to give the nitro amine (XIII) 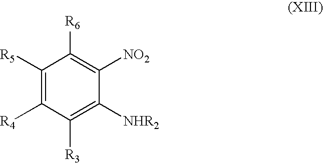
followed by reduction of the nitro group by conventional means, for example using hydrogen and a palladium catalyst, iron and an organic acid e.g. acetic acid or with sodium dithionite.

When compounds of formula (I) contain an asymmetric centre the specific enantiomers arising there from may be obtained by conventional procedures. For example using preparative high performance liquid chromatography (HPLC) with a chiral stationary phase.

Physiologically acceptable acid addition salts of the compounds of formula (I) wherein one of the groups R₁ to R₅ contain a basic nitrogen atom may be prepared by conventional procedures. Thus by addition of a solution of the inorganic or organic acid in a suitable solvent e.g. an alkanol or an ether to a solution of the free base in a solvent such as an alkanol e.g. methanol or an ether e.g. diethyl ether or tetrahydrafuran.

The compounds of formula (III), (IV), (V) (IX), (X) and (XII) are either known compounds or may be prepared by analogous methods to those preparing the known compounds.

The following examples are illustrative of the present invention and are not to be construed as a limitation of the scope of the invention.

### Intermediate 1. 5-Methyl-[1,2,5]oxadiazolo[3,4-d]pyrimidin-7-ol

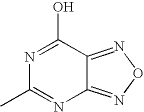

A solution of 4-amino-6-hydroxy-2-methyl-5-nitrosopyrimidine (20 g, 130 mmol) in acetic acid (800 ml) was treated portionwise with lead tetraacetate (86 g, 195 mmol) and stirred overnight at room temperature. The solvent was evaporated in vacuo and the residue partitioned between ethyl acetate and water. The organic phase was washed with water and saturated sodium bicarbonate solution, dried over magnesium sulphate and concentrated in vacuo. Trituration of the residue with dichloromethane afforded the title compound (6.33 g, 32%). ¹H NMR (DMSO-d₆); 12.65 (1H, brs), 2.38 (3H, s).

### EXAMPLE 1

### 4-(1-Ethyl-1H-benzoimidazol-2-yl)-furazan-3-ylamine

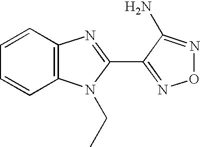

A mixture of N-ethylbenzene-1,2-diamine (212 mg, 1.56 mmol) (Weidenhagan et. al., Chem. Ber.; 1942, 75, 1936) and 4-aminofurazan-3-carboxylic acid (200 mg, 1.5 mmol) was added to polyphosphoric acid (2 ml) and the mixture was stirred at 180° C. for 1 hour. After cooling to room temperature the reaction mixture was added to saturated potassium carbonate solution and extracted with ethyl acetate. The organic phase was concentrated in vacuo and the residue purified by silica gel chromatography eluting with 10% ethyl acetate in hexanes, to afford the title compound, (20 mg, 6%); MS (AP+) m/e 230 [M+H]⁺.

### EXAMPLE 2

### 4-(1H-Benzoimidazol-2-yl)-furazan-3-ylamine

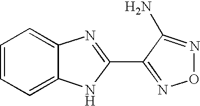

To a solution of (1H-benzoimidazol-2-yl)acetonitrile (5.0 g, 31.8 mmol) in hydrochloric acid (2N, 100 ml) and methanol (50 ml) was added sodium nitrite (4.3 g, 63.6 mmol) portionwise. After stirring at room temperature for 3 hours, the mixture was basified to pH10 with 50% sodium hydroxide solution, hydroxylamine (20 ml) was added and the solution refluxed for 18 hours. The mixture was cooled and the precipitate collected to afford the title compound (1.8 g, 22%); MS (AP+) m/e 202 [M+H]⁺.

### EXAMPLE 3

### 4-[2-(Amino-furazan-3-yl)-benzoimidazol-1-yl]-piperidine-1-carboxylic acid tert-butyl ester

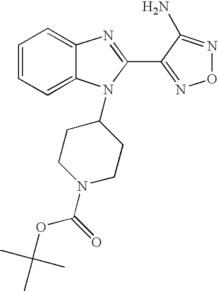

A stirred solution of the product of Example 2 (600 mg, 3 mmol), 4-hydroxy-piperidine-1-carboxylic acid tert-butyl ester (600 mg, 3 mmol) and triphenyl-phosphine (1.18 g, 4.5 mmol) in tetrahydrofuran (7 ml) at 0° C. was treated with diethyl azodicarboxylate (0.71 ml, 4.5 mmol). The solution was stirred at room temperature for 4 hours and the solvent was removed in vacuo. The residue was purified by silica gel chromatography eluting with 0.880 ammonia:ethanol:hexane (1:9:40) to afford the title compound (160 mg, 13%); MS (ES+) m/e 385 [M+H]⁺.

### EXAMPLE 4

### 4-(1-Piperidin-4-yl-1H-benzoimidazol-2-yl)-furazan-3-ylamine

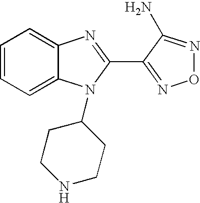

The product of Example 3 (160 mg, 0.41 mmol) was stirred in trifluoroacetic acid (2 ml) and dichloromethane (4 ml) at room temperature for 3 hours and the solution was then co-evaporated with dichloromethane. The residue was purified by silica gel chromatography eluting with 0.880 ammonia:ethanol:hexane (1:9:200), to afford the title compound (50 mg, 42%); MS (ES+) m/e 285 [M+H]⁺.

### EXAMPLE 5

### 4-[1-(2-Amino-ethyl)-1H-benzoimidazol-2-yl]-furazan-3-ylamine

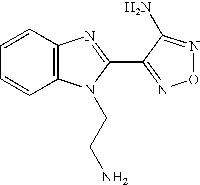

To a solution of the product of Example 2 (250 mg, 1.24 mmol) in N,N-dimethylformamide (2 ml) at 0° C. was added sodium hydride (60% suspension in mineral oil, 55 mg, 1.37 mmol). The mixture was stirred at 0° C. for 15 minutes then treated with additional sodium hydride (55 mg, 1.37 mmol) followed by 2-chloroethylamine hydrochloride (160 mg, 1.37 mmol) and the mixture was stirred at 70° C. overnight. On cooling, the mixture was poured into saturated aqueous sodium hydrogen carbonate solution. The mixture was extracted with ethyl acetate (2×) and the organic phase washed with saturated aqueous sodium chloride solution, dried over magnesium sulphate and concentrated in vacuo. The residue was purified by silica gel chromatography eluting with 5% methanol in dichloromethane, triturated with ether, filtered and dried in vacuo to afford the title compound (80 mg, 26%); MS (AP+) m/e 245 [M+H]⁺.

### EXAMPLE 6

### 4-[1-(2-Amino-1-methylethyl)-1-H-benzoimidazol-2-yl]furazan-3-ylamine

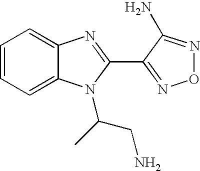

### Step 1. 2-[2-(4-Amino-furazan-3-yl)-benzoimidazol-1-yl]-propionitrile

The title compound was prepared from the product of Example 2 and 2-chloropropionitrile using the method of Example 5; MS (AP+) m/e 255 [M+H]⁺.

### Step 2. 4-[1-(2-Amino-1-methylethyl)-1H-benzoimidazol-2-yl]furazan-3-ylamine

The product from Step 1 (150 mg, 0.59 mmol) in dry tetrahydrofuran (5 ml) was treated under argon with a 1.0M borane-tetrahydrofuran complex (1.24 ml, 1.24 mmol) and heated under reflux for 3 hours. After cooling to room temperature, methanol was added dropwise, followed by 2M hydrochloric acid, and the mixture was heated under reflux for 90 minutes. After cooling to room temperature, the solvent was removed in vacuo and the residue partitioned between 10% aqueous sodium hydroxide solution and dichloromethane. The organic phase was separated, dried over magnesium sulphate and concentrated in vacuo to afford the title compound (40 mg, 27%); MS (ES+) m/e 259 [M+H]⁺.

### EXAMPLE 7

### 4-(5-Bromo-1-ethyl-1H-benzoimidazol-2-yl)furazan-3-ylamine

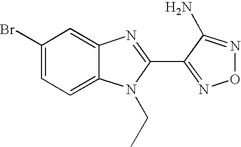

### Step 1. (4-Bromo-2-nitrophenyl)ethylamine

5-Bromo-2-fluoronitrobenzene (1.2 g, 5.7 mmol) in ethanol (4 ml) was cooled to 0° C. and treated with a 70% solution of ethylamine in water (4 ml). After warming to room temperature, the mixture was stirred for 30 minutes and the title compound (1.25 g, 85%) was collected and dried in vacuo; ¹H NMR (CDCl₃) 8.31 (1H, d, J 1 Hz), 7.95 (1H, br s), 7.49 (1H, dd, J 8.5, 1 Hz), 6.75 (1H, d. J 8.5 Hz), 3.34 (2H, m), 1.37 (3H, t, 8.5 Hz).

### Step 2. 4-Bromo-N¹-ethylbenzene-1,2-diamine

The product from Step 1 (1.25 g, 4.8 mmol) was treated with a solution of tin (II) chloride (3.20 g, 16.9 mmol) in concentrated hydrochloric acid (4.25 ml) and stirred at 50° C. for 1 hour. After cooling to room temperature, the reaction mixture was poured into 10% sodium hydroxide solution, extracted into ethyl acetate, and the organic phase washed with saturated aqueous sodium chloride solution, dried over magnesium sulphate and evaporated in vacuo to afford the title compound (800 mg, 76%); ¹H NMR (DMSO-d₆) 6.66 (1H, d, J 1 Hz), 6.57 (1H, dd, J 8.5, 1 Hz), 6.29 (1H, d, J 8.5 Hz), 4.81 (2H, br s), 4.45 (1H, br m), 3.00 (2H, m), 1.19 (3H, t, J8.5 Hz)

### Step 3. 4-(5-Bromo-1-ethyl-1H-benzoimidazol-2-yl)furazan-3-ylamine

The product from Step 2 (500 mg, 2.9 mmol) and Intermediate 1 (290 mg, 1.9 mmol) in glacial acetic acid (5 m[) were heated under reflux for 3 hours. After cooling to room temperature, the acetic acid was removed in vacuo and the residue triturated with dichloromethane, filtered and dried in vacuo to afford the title compound (100 mg, 17%); MS(AP+) m/e 308/310 [M+H]⁺.

The following examples were prepared from 2-fluoronitrobenzene and the appropriate amine by the general method described in Example 7, Steps 1-3.

| | Example | Amine | Characterisation |
|---|---|---|---|
| 8 | 4-(1-Cyclopropyl-1 H- | Cyclopropylamine | MS (AP+) m/e |
| | benzoimidazol-2-yl)-furazan-3-ylamine | | 242 [M + H]⁺ |
| 9 | 4-(1-Cyclohexyl-1 H- | Cyclohexylamine | MS (AP+) m/e |
| | benzoimidazol-2-yl)-furazan-3- | | 284 [M + H]⁺ |
| | ylamine | | |
| 10 | 4-(1-Cyclopropylmethyl-1 H- | C-Cyclopropylmethylamine | MS (AP+) m/e |
| | benzoimidazol-2-yl)-furazan-3- | | 256 [M + H]⁺ |
| | ylamine | | |
| 11 | 4-[1-(2-Dimethylamino-ethyl)-1 | N¹,N¹-Dimethyl-ethane- | MS (AP+) m/e |
| | H-benzoimidazol-2-yl]-furazan- | 1,2-diamine | 273 [M + H]⁺ |
| | 3-ylamine | | |
| 12 | 4-[1-(2-Dimethylamino-1- | N¹,N¹-Dimethyl-propane- | MS (AP+) m/e |
| | methyl-ethyl)-1 H- | 1,2-diamine | 287 [M + H]⁺ |
| | benzoimidazol-2-yl]-furazan-3- | | |
| | ylamine | | |
| 13 | 4-[1-(3-Chlorophenyl)-1-H- | 3-Chloroaniline | MS (AP+) m/e |
| | benzoimidazol-2-yl]furazan-3- | | 312/314 [M + H]⁺ |
| | ylamine | | |
| 14 | 4-[1-(4-Chlorophenyl)-1H- | 4-Chloroaniline | MS (AP+) m/e |
| | benzoimidazol-2-yl]furazan-3- | | 312/314 [M + H]⁺ |
| | ylamine | | |

The following examples were prepared from Intermediate 1 and the appropriate diamine by the general method described in Example 7, Step 3.

| | Example | 1,2-Diamine | Characterisation |
|---|---|---|---|
| 15 | 4-(6-Chloro-1-ethyl-1H- | 4-Chloro-N²-ethyl- | MS (AP−) m/e |
| | benzoimidazol-2-yl)furazan-3- | benzene-1,2-diamine | 262/264 [M − H]⁻. |
| | ylamine | (Davoll et. al.; J. Chem. | |
| | | Soc. 1960, 314) | |
| 16 | 4-(1-Ethyl-5-methoxy-1H- | N¹-ethyl-4-methoxy- | MS (ES+) m/e |
| | benzoimidazol-2-yl)-furazan-3- | benzene-1,2-diamine | 260 [M + H]⁺ |
| | ylamine | (Tanaka et. al. Chem. | |
| | | Pharm. Bull. 1981, 29, 1876). | |
| 17 | 4-(4-Nitro-1 H-benzimidazol-2- | 3-Nitro-benzene-1,2- | MS (ES+) m/e |
| | yl)-furazan-3-ylamine | diamine | 245 [M + H]⁺. |
| 18 | 4-(5,6-dihydro-4H- | 1,2,3,4- | MS (ES+) 242 |
| | imidazo[4,5,1-ij]quinolin-2-yl)- | tetrahydroquinoline-8- | [M + H]; |
| | furazan-3-ylamine | amine | |
| 19 | 4-(1-phenyl-1H-benzimidazol- | N-phenylbenzene-1,2- | MS (ES+) 278 |
| | 2-yl)-furazan-3-ylamine | diamine | [M + H]; |
| 20 | 4-(5-chloro-6-fluoro-1H- | 3-chloro-4-fluoro-1,2- | MS (ES+) 254 |
| | benzimidazol-2-yl)-furazan-3- | phenylenediamine | [M + H]; |
| | ylamine | | |
| 21 | 4-(1-methyl-1H-benzimidazol- | N-methylbenzene-1,2- | MS (ES+) 215 |
| | 2-yl)-furazan-3-ylamine | diamine | [M + H]; |
| 22 | 4-(6-fluoro-1H-benzimidazol-2- | 4-fluorobenzene-1,2- | MS (ES+) 220 |
| | yl)-furazan-3-ylamine | diamine | [M + H]; |
| 23 | 4-(1-pyridin-3-yl-1H- | N-pyridin-3-ylbenzene- | MS (ES+) 279 |
| | benzimidazol-2-yl)-furazan-3- | 1,2-diamine | [M + H]; |
| | ylamine | | |

### EXAMPLE 24

### [2-(4-Amino-furazan-3-yl)-1-ethyl-1H-benzoimidazol-5-yl]-ethanone

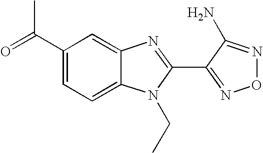

To a solution of the product of Example 7 (200 mg, 0.649 mmol) in anhydrous toluene (15 ml), was added (1-ethoxyvinyl)tributyl tin (0.241 ml, 0.714 mmol) followed by bis(triphenylphosphine)palladium (II) dichloride (45 mg, 0.0649 mmol), and the reaction heated under reflux for 16 hours under an argon atmosphere. The solvent was evaporated in vacuo and the residue dissolved in methanol (5 ml) and 2N hydrochloric acid (1.95 ml) and stirred for 15 minutes at room temperature. The solvent was evaporated in vacuo, neutralised with saturated sodium bicarbonate solution, diluted with dichloromethane, and the resulting precipitate collected by filtration. The crude product was triturated with hot methanol, and dried in vacuo to afford the title product (45 mg, 26%). MS (ES+) m/e 272 [M+H]⁺.

### EXAMPLE 25

### 2-(4-Amino-furazan-3-yl)-1-ethyl-1H-benzoimidazole-5-carbaldehyde

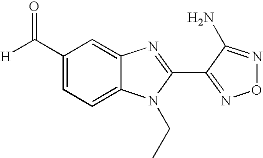

To a solution of the product of Example 7 (180 mg, 0.584 mmol) in anhydrous N,N-dimethylformamide (10 ml) was added bis(triphenylphosphine)palladium (II) dichloride (40 mg, 0.0584 mmol), followed by sodium formate (120 mg, 1.752 mmol), and the reaction was maintained under atmospheric pressure of carbon monoxide and heated at 100° C. for 16 hours. The solvent was evaporated in vacuo, and the residue partitioned between ethyl acetate and saturated sodium bicarbonate solution. The organic phase was then washed with water (×3), dried over anhydrous sodium sulphate, and evaporated in vacuo to yield a residue. The crude residue was purified by silica gel chromatography eluting with a mixture of ethyl acetate/hexane (1:2.5) to afford the title product (22 mg, 15%). MS (ES+) m/e 258 [M+H]⁺.

### EXAMPLE 26

### [2-(4-Amino-furazan-3-yl)-1-ethyl-1H-benzoimidazol-5-yl]-methanol

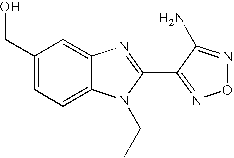

To a solution of the product of Example 25 (21 mg, 0.082 mmol) in anhydrous methanol was added sodium borohydride (3.4 mg, 0.090 mmol). After stirring for 2 hours at room temperature, the solvent was evaporated in vacuo and the residue dissolved in dichloromethane, and the organic phase washed with saturated sodium bicarbonate solution, then water and dried over anhydrous sodium sulphate. The solvent was then evaporated in vacuo to afford the title product (19 mg, 90%). MS (ES+) m/e 260 [M+H]⁺.

### EXAMPLE 27

### 2-(4-Aminofurazan-3-yl)-3-ethyl-3H-benzoimidazol-5-ol

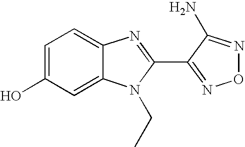

### Step 1. [2-(3-Fluoro-4-nitrophenoxymethoxy)ethyl]trimethylsilane

3-Fluoro-4-nitrophenol (2.8 g, 18.3 mmole) in dry N,N-dimethylformamide (10 ml) was cooled to 0° C. and treated with a 60% suspension of sodium hydride in mineral oil (810 mg, 20.2 mmol). The mixture was stirred at room temperature for 15 minutes. 2-(Trimethylsilyl)ethoxymethyl chloride (3.6 ml, 20.2 mmol) was added dropwise and the mixture stirred for 18 hours at room temperature. The reaction mixture was poured into water, extracted (×3) into dichloromethane and the organic phase washed with saturated aqueous sodium chloride solution, dried over magnesium sulphate and concentrated in vacuo to afford the title compound (5.0 g, 95%); ¹H NMR (CDCl₃) 8.07 (1H, t, J 8.5 Hz), 6.96 (2H, m), 5.28 (2H, s), 3.75 (2H, t, J 8.5 Hz), 0.93 (2H, t, J 8.5 Hz).

### Step 2. Ethyl-[2-nitro-5-(2-trimethylsilanyl-ethoxymethoxy)-phenyl]-amine

The product from Step 1 (5.0 g, 17.4 mmole) in ethanol (12 ml) was treated with a 70% solution of ethylamine in water (12 ml) and heated at 60° C. for 18 hours. After cooling to room temperature, the solvent was removed in vacuo and the residue dissolved in dichloromethane, washed with water and saturated aqueous sodium chloride solution, dried over magnesium sulphate and concentrated in vacuo to afford the title compound (4.6 g, 85%); MS (ES+) m/e 313 [M+H]⁺.

### Step 3. N²-Ethyl-4-(2-trimethylsilanylethoxymethoxy)benzene-1,2-diamine

The product from Step 2 (4.6 g, 14.7 mmole) in ethanol (150 ml) was hydrogenated for 18 hours in the presence of 10% palladium on carbon. After filtration of the catalyst through Kieselguhr, the filtrate was concentrated in vacuo to afford the title compound (4.1 g, 100%); MS (AP+) m/e 283 [M+H]⁺.

### Step 4. 1-Ethyl-6-(2-trimethylsilanylethoxymethoxy)-1H-benzoimidazole-2-carbonitrile

The product of Step 3 (1.47 g, 5.2 mmol) and ethyl cyanoacetate (0.83 ml, 7.9 mmol) was heated to 190° C. for 20 minutes. After cooling to room temperature the mixture was purified by silica gel chromatography eluting with 2560% ethyl acetate in hexanes, to afford the title compound, (300 mg, 17%), MS (ES+) m/e 332 [M+H]⁺.

### Step 5. 2-(4-Aminofurazan-3-yl)-3-ethyl-3-H-benzoimidazol-5-ol

The title compound was prepared from the product of Step 4 using the method described in Example 2; MS (ES+) m/e 246 [M+H]⁺.

### EXAMPLE 28

### 2-(4-Aminofurazan-3-yl)-1-ethyl-1H-benzoimidazol-5-ol

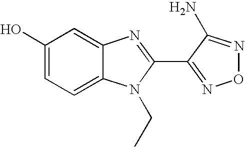

The title compound was prepared from 4-chloro-3-nitrophenol using the general methods described in Example 27 Steps 1-5; MS (ES+) m/e 246 [M+H]⁺.

### EXAMPLE 29

### 4-(1-Ethyl-4-nitro-1H-benzimidazol-2-yl)-furazan-3-ylamine

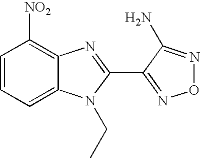

The title compound was prepared from the product of Example 17 and ethyl iodide using the method of Example 5; MS (ES+) m/e 275 [M+H]⁺.

### EXAMPLE 30

### 4-(1-Ethyl-1H-benzoimidazol-2-yl)-1H-pyrazol-3-ylamine

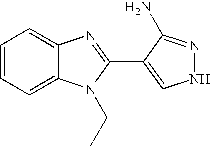

### Step 1. (1-Ethyl-1H-benzoimidazol-2-yl)-acetonitrile

A solution of (1H-benzoimidazol-2-yl)-acetonitrile (1.57 g, 0.75 mmol) in N,N-dimethylformamide (20 ml) containing cesium carbonate (3.3 g, 10 mmol) was treated with ethyl iodide (1.56 g, 0.8 ml, 10 mmol). After stirring at room temperature for 5 hours the mixture was filtered through filter aid and the filter pad washed with ethyl acetate. The filtrates were combined, washed with water and brine, dried and concentrated in vacuo. The residue was purified by silica gel chromatography eluting with ethyl acetate, to afford the title compound, (610 mg, 33%); MS (AP+) m/e 186 [M+H]⁺.

### Step 2. Dimethylamino-(1-ethyl-1H-benzoimidazol-2-yl)-acrylonitrile

The product of Step 1 (1.86 g, 10 mmol) and N,N-dimethylformamide dimethylacetal (1.19 g, 1.3 ml, 10 mmol) in ortho-xylene (10 ml) was heated under reflux for 1 hour. After cooling, the solution was concentrated and the residue co-evaporated with toluene (×3) to afford the title compound (2.06 g, 86%); MS (ES+) m/e 241 [M+H]⁺.

### Step 3. 4-(1-Ethyl-1H-benzoimidazol-2-yl)-1H-pyrazol-3-ylamine

The product of Step 2 (381 mg, 1.8 mmol) and hydrazine hydrate (180 mg, 3.6 mmol) in methanol (10 ml) was heated under reflux for 5 hours. After cooling, the reaction mixture was concentrated in vacuo and the residue purified by silica gel chromatography eluting with 10% methanol in chloroform, to afford the title compound (203 mg, 57%); MS (ES+) m/e 200 [M+H]⁺.

### EXAMPLE 31

### 4-[1-(2,2,2-Trifluoroethyl)-1H-benzimidazol-2-yl]-furazan-3-ylamine

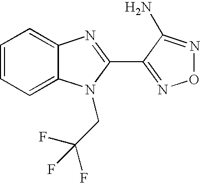

A solution of 1,1,1-difluoro-2-iodoethane (1 mL) and the product of Example 2 (0.056 g, 0.28 mmol) in N,N-dimethylformamide (3 mL) was treated with cesium carbonate (560 mg) and the mixture heated to 100° C. for 2 hours. After cooling the reaction mixture was concentrated to dryness and diluted with ethyl acetate and water. The organic layer was separated, dried over magnesium sulfate and concentrated in vacuo and the residue was purified by silica gel chromatography to yield the title compound (26 mg, 33%); MS (ES+) m/e 284 [M+H]⁺.

### EXAMPLE 32

### 4-{1-[3-(4-Methylpiperazin-1-yl)propyl]-1H-benzimidazol-2-yl}-furazan-3-ylamine

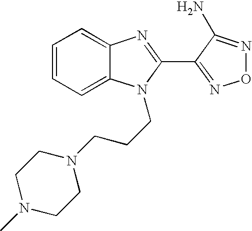

### Step 1. [3-(4-Methylpiperazin-1-yl)propyl]-(2-nitrophenyl)amine

2-Fluoronitrobenzene (1.0 g, 7.1 mmol) in tetrahydrofuran (25 ml) was treated with 1-(3-aminopropyl)-4-methylpiperazine (1.6 g, 10 mmol) and potassium carbonate (1.0 g, 7.8 mmol). The resulting mixture was stirred at room temperature for 16 h, and the mixture diluted with water and ethyl acetate. The organic extracts were collected and dried over magnesium sulfate, the filtrate was concentrated ni vacuo and the residue was purified by silica gel chromatography to yield the title compound (1.5 g, 76%); ¹H NMR (CDCl₃) δ 8.28 (1H, br t), 8.20 (1H, d), 7.45 (1H, dd), 6.92 (1H, d), 6.65 (1H, dd), 3.42 (2H, q), 2.36-2.64 (10H, br m), 2.34 (3H, s), 1.92 (2H, m).

### Step 2. N-[3-(4-Methylpiperazin-1-yl)propyl]benzene-1,2-diamine

The product from Step 1 (1.5 g, 5.4 mmol) in methanol (40 ml) containing 10% palladium on carbon (200 mg) was hydrogenated at 45 psi. The mixture was filtered through silica gel and the filtrate was concentrated in vacuo. The resulting dark oil was stirred vigorously in diethyl ether, allowed to settle and the supernatant liquid was decanted and concentrated to an oil to afford the title compound (0.70 g, 52%); ¹H NMR (DMSO-d₆) 6.40-6.60 (4H, br m), 4.45 (2H, br s), 3.38 (1H, br s), 3.02 (2H, t), 2.25-2.50 (10H, br m), 2.18 (3H, s), 1.75 (2H, m).

### Step 3. 4-{1-[3-(4-Methylpiperazin-1-yl)propyl]-1H-benzimidazol-2-yl}-furazan-3-ylamine

The product from Step 2 (270 mg, 1.1 mmol) and Intermediate 1 (80 mg, 0.5 mmol) in glacial acetic acid (5 ml) were heated under reflux for 3 hours. After cooling to room temperature, the acetic acid was removed in vacuo and the residue triturated with dichloromethane, filtered and dried in vacuo to afford the title compound (84 mg, 49%); MS (ES+) m/e 342 [M+H]⁺.

### EXAMPLE 33

### 4-{1-[(1-Ethylpyrrolidin-2-yl)methyl]-1H-benzimidazol-2-yl}-furazan-3-ylamine

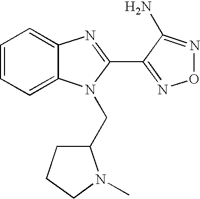

The title compound was prepared from 2-fluoronitrobenzene and 2-(aminomethyl)-1-ethylpyrrolidine by the general method described in Example 32, Steps 1-3. MS (ES+) m/e 313 [M+H]⁺.

### EXAMPLE 34

### [2-(4-Amino-furazan-3-yl)-1-ethyl-1H-benzimidazol-4yl]-ethylamine

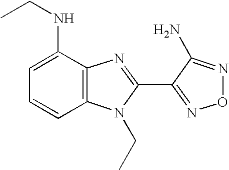

### Step 1. N,N′-Diethyl-2-nitrobenzene-1,3-diamine

2,6-Difluoronitrobenzene (1.0 g, 6.3 mmol) was treated with 70% ethylamine in water (10 mL) and stirred at room temperature for 3 h. The mixture was diluted with water and the precipitate was collected washed with water and dried to afford the title compound (1.3 g, 98%); ¹H NMR (DMSO-d₆) 8.42 (2H, br t), 7.22 (1H, t), 6.00 (2H, d), 3.25 (4H, m), 1.12 (6H, t).

### Step 2. N′,N³-Diethylbenzene-1,2,3-triamine

The product from Step 1 (0.3 g, 1.4 mmol) in methanol (15 ml) containing 10% palladium on carbon (6 mg) was hydrogenated at 45 psi. The mixture was filtered through silica gel and the filtrate was concentrated in vacuo. The residue was triturated with hexanes and the solid was collected to afford the title compound (0.16 g, 64%); ¹H NMR (CDCl₃) 6.85 (1H, t), 6.38 (2H, d), 2.60-3.40 (8H, br m), 1.16 (6H, t).

### Step 3. [2-(4-Amino-furazan-3-yl)-1-ethyl-1H-benzimidazol-4yl]-ethylamine

The product from Step 2 (92 mg, 0.51 mmol) and Intermediate 1 (62 mg, 0.4 mmol) in glacial acetic acid (2 ml) were heated under reflux for 0.5 hours. After cooling to room temperature, the acetic acid was removed in vacuo and the residue dissolved in ethyl acetate and washed with solution of dilute sodium bicarbonate. The organics were dried over magnesium sulfate and the filtrate was concentrated in vacuo. The residue was purified by silica gel chromatography to afford the title compound (12 mg, 10%); MS (ES+) m/e 273 [M+H]⁺.

### EXAMPLE 35

### 4-(1-Propyl-1H-benzimidazol-2-yl)-furazan-3-ylamine

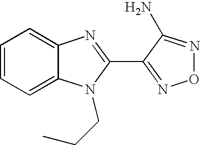

A solution of 1-iodopropane (0.05 mL, 0.5 mmol) and the product of Example 2 (0.051 g, 0.25 mmol) in N,N-dimethylformamide (2 mL) was treated with cesium carbonate (82 mg) and the mixture stirred at room temperature for 3 h. The reaction was concentrated in vacuo and the residue partitioned between ethyl acetate and water. The organic extract was separated, dried over magnesium sulfate and the filtrate was concentrated in vacuo. The residue was triturated with diethyl ether to give the title compound (33 mg, 54%); MS (ES+) m/e 244 [M+H]⁺.

The following examples were prepared from the product of Example 2 and the appropriate halide by the general method described in Example 35.

| | Example | Halide | Characterisation |
|---|---|---|---|
| 36 | 4-[1-(Cyclobutylmethyl)-1H- | Bromomethyl- | MS (ES+) m/e |
| | benzimidazol-2-yl]-furazan-3- | cyclobutane | 270 [M + H]⁺ |
| | ylamine | | |
| 37 | 4-(1-Butyl-1H-benzimidazol-2- | 1-Bromobutane | MS (ES+) m/e |
| | yl)-furazan-3-ylamine | | 270 [M + H]⁺ |

### EXAMPLE 38

### 4-(1-Ethyl-5-fluoro-1H-benzimidazol-2-yl)-furazan-3-ylamine

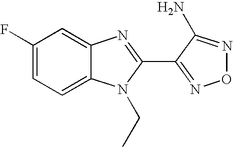

### Step 1. (2-Amino-5-fluorophenyl)-carbamic Acid Tert-Butyl Ester

A mixture of 4-fluorobenzene-1,2-diamine (6.9 g, 55 mmol) and di(tert-butyl) dicarbonate (12 g, 55 mmol) was warmed to 50° C. and stirred until homogeneous (10 min). The mixture was patitioned between ethyl acetate and water. The organic extracts were combined, washed with water and brine, dried over magnesium sulfate and concentrated in vacuo. The residue was triturated with hexanes to afford the title compound (9.1 g, 73%); ¹H NMR (DMSO-₆) 8.25 (1H, brs), 7.11 (1H, m), 6.48 (1H, m), 6.31 (1H, m), 5.15 (2H, br s), 1.51 (9H, s).

### Step 2. (2-Ethylamino-5-fluorophenyl)-carbamic Acid Tert-Butyl Ester

A solution of the product from Step 1 (9.1 g, 40 mmol) in tetrahydrofuran (100 mL) was treated with solid potassium tert-butoxide (5.37 g, 48 mmol). Iodoethane (3.8 ml, 48 mmol) was added dropwise and the mixture was stirred at room temperature for 1 h. The reaction mixture was diluted with ethyl acetate and water and the organic layer was separated, washed with water and brine and dried over magnesium sulfate. The solvent was concentration in vacuo and the residue recrystalised from diethyl ether to afford the title compound (3.9 g, 38%); ¹H NMR (DMSO-d₆) 6.85 (1H, m), 6.48 (1H, m), 6.29 (1H, m), 5.21 (1H, brs), 3.67 (2H, m), 3.14 (1H, m), 1.38 (9H, s), 1.02 (3H, t).

### Step 3. 4-(1-Ethyl-5-fluoro-1H-benzimidazol-2-yl)-furazan-3-ylamine

The product from Step 2 (2.139, 8.6 mmol) and Intermediate 1 (1.05 g, 6.9 mmol) in glacial acetic acid (10 ml) were heated to reflux for 3 hours. After cooling to room temperature, the acetic acid was removed in vacuo and the residue triturated with dichloromethane, filtered and dried in vacuo to afford the title compound (0.919, 43%); MS (ES+) m/e 248 [M+H]⁺; ¹H NMR (DMSO-d₆) 7.90 (1H, m), 7.66 (1H, m), 7.36 (1H, m), 6.99 (2H, br s), 4.72 (2H, q), 1.20 (3H, t).

### EXAMPLE 39

### 4-(1-Ethyl-6-fluoro-1H-benzimidazol-2-yl)-furazan-3-ylamine

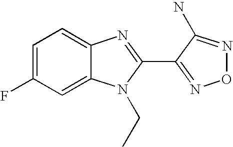

The title compound was prepared from 2,4-difluoronitrobenzene and aqueous ethyl amine using the procedures described in Example 32, Step 1-3; MS (ES+) m/e 248 [M+H]+;]; ¹H NMR (DMSO-d₃) 7.88 (1H, dd), 7.76 (1H, m), 7.28 (1H, m), 6.98 (2H, br s), 4.71 (2H, q), 1.38 (3H, t), 1.38 (3H, t).

### EXAMPLE 40

### 4-{1-[(1S)-1-Phenylethyl]-1H-benzimidazol-2-yl}-furazan-3-ylamine

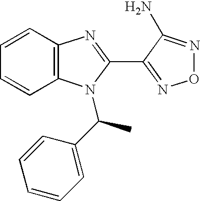

The title compound was prepared from 2-fluoronitrobenzene and (S)-1-phenylethylamine using the procedures described in Example 32, Step 1-3; ¹H NMR (DMSO-d₆) 7.84 (1H, d), 7.36 (6H, m), 7.17 (2H, m), 7.09 (1H, m), 6.06 (2H, brs), 1.38 d).

The following examples were obtained from commercial sources as indicated:

| | Example | Source |
|---|---|---|
| 41 | 6-[2-(4-Amino-furazan-3-yl)- | ChemBridge Corporation, 16981 |
| | benzoimidazole-1-ylmethyl]- | Via Tazon, Suite G, San Diego |
| | [1,3,5]triazine-2,4-diamine | CA 92127 USA |
| 42 | [2-(4-Amino-furazan-3-yl)- | ChemBridge Corporation, 16981 |
| | benzoimidazole-1-yl]- | Via Tazon, Suite G, San Diego |
| | acetonitrile | CA 92127 USA |
| 43 | 4-(1-Allyl-1 H-benzoimidazol-2- | ChemBridge Corporation, 16981 |
| | yl)-furazan-3-ylamine | Via Tazon, Suite G, San Diego |
| | | CA 92127 USA |
| 44 | 4-(1-Isopropyl-1 H- | ChemBridge Corporation, 16981 |
| | benzoimidazol-2-yl)-furazan- | Via Tazon, Suite G, San Diego |
| | 3-ylamine | CA 92127 USA |
| 45 | 4-(1-Prop-2-ynyl-1 H- | ChemBridge Corporation, 16981 |
| | benzoimidazol-2-yl)-furazan- | Via Tazon, Suite G, San Diego |
| | 3-ylamine | CA 92127 USA |
| 46 | 4-[1-(2-Vinyloxyethyl)-1 H- | ChemBridge Corporation, 16981 |
| | benzoimidazol-2-yl]-furazan- | Via Tazon, Suite G, San Diego |
| | 3-ylamine | CA 92127 USA |
| 47 | 4-(1 H-benzoimidazol-2-yl)- | ChemDiv Inc., P.O. Box 90639, |
| | furazan-3-ylamine | San Diego CA 92169 USA |

### EXAMPLE 48

### 2-(4-Amino-furazan-3-yl)-1-(1,2,3,4-tetrahydro-7-isoquinolinyl)-1H-benzimidazol-6-ol

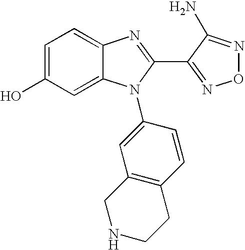

The title compound was prepared from 3-fluoro-4-nitrophenol and 7-amino-3,4-dihydro-1H-isoquinoline-2-carboxylic acid t-butyl ester (WO99/14197) by the method of Example 27 Steps 2-5, followed by treatment with TFA in CH₂Cl₂ to remove BOC protecting group. MS (ES+) m/e 349 [M+H]⁺.

### EXAMPLE 49

### 1,1-Dimethylethyl 7-[2-(4-amino-furazan-3-yl)-6-fluoro-1H-benzimidazol-1-yl]-3,4-dihydro-2(1H)-isoquinolinecarboxylate

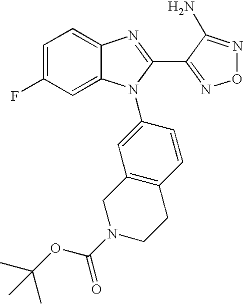

The title compound was prepared from 2,4-fluoro-nitrobenzene and 7-amino-3,4-dihydro-1H-isoquinoline-2-arboxylic acid t-butyl ester (WO99/14197) by the method of Example 27 Steps 2-5. MS (ES+) m/e 451 [M+H]⁺.

### EXAMPLE 50

### 4-[1-Ethyl-6-(methyloxy)-1H-benzimidazol-2-yl]-furazan-3-ylamine

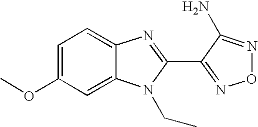

### Step 1. 4-Chloro-2-(ethylamino)-nitrobenzene

Ethylamine (2 M in THF, 50 mL, 100 mmol) was added to a solution of 2,4-dichloro-nitrobenzene (9.6 g, 50 mmol) and triethylamine (7.6 mL, 55 mmol) in THF (10 mL). The solution was heated to 65° C. for 6 days. The reaction mixture was cooled, poured into H₂O and extracted with EtOAc. The organic extracts were washed with brine, dried (MgSO₄), filtered and concentrated. The crude material was purified by column chromatography (10-80% EtOAc in hex) to give the title compound (6.41 g, 64%). MS (ES+) m/e 201 [M+H]⁺.

### Step 2. 2-(Ethylamino)-4-methoxy-nitrobenzene

The product from Step 1 (500 mg, 2.5 mmol) was dissolved in MeOH (5 mL). Sodium methoxide (2 mL of a 25% solution in methanol) was added and the mixture was heated to 60° C. for 3 days. The reaction mixture was cooled, poured into H₂O and extracted with EtOAc. The organic extracts were washed with brine, dried (MgSO₄), filtered and concentrated to give the title compound (465 mg, 95%). MS (ES+) m/e 197 [M+H]⁺.

### Step 3. 4-[1-Ethyl-6-(methyloxy)-1H-benzimidazol-2-yl]-furazan-3-ylamine

The title compound was obtained from the product of Step 2 by the general method of Example 27, Steps 3-5. MS (ES+) m/e 260 [M+H]⁺.

### EXAMPLE 51

### 2-(4-amino-furazan-3-yl)-1-ethyl-N,N-dimethyl-1H-benzimidazol-6-amine

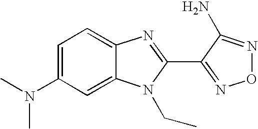

### Step 1. 2-(Ethylamino)-4-dimethylamino-nitrobenzene

Dimethylamine hydrochloride (410 mg, 7.5 mmol), triethylamine (1.0 mL, 7.5 mmol) and the product from Example 50, Step 1 (500 mg, 2.5 mmol) were heated to 200° C. in DMF (5 mL) in a microwave reactor for 15 min. The reaction mixture was cooled, poured into H₂O and extracted with EtOAc. The organic extracts were washed with brine, dried (MgSO₄), filtered and concentrated. The residue was chromatographed (5-95% EtOAc in hex) to give the title compound (450 mg, 86%). MS (ES+) m/e 210 [M+H]⁺.

### Step 2. 2-(4-Amino-furazan-3-yl)-1-ethyl-N,N-dimethyl-1H-benzimidazol-6-amine

The title compound was obtained from the product of Step 1 by the general method of Example 27, Steps 3-5. MS (ES+) m/e 274 [M+H]⁺.

### EXAMPLE 52

### 4-[1-Ethyl-6-(phenyloxy)-1H-benzimidazol-2-yl]-furazan-3-ylamine

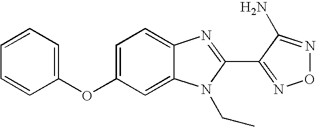

### Step 1. 2-(Ethylamino)-4-fluoro-nitrobenzene

The title compound was prepared from 2,4-difluoronitrobenzene and ethylamine by the method of Example 50, Step 1. MS (ES+) m/e 185 [M+H]⁺.

### Step 2. 2-(Ethylamino)-4-phenyloxy-nitrobenzene

Phenol (560 mg, 6.0 mmol) in NMP (3 mL) was added to a slurry of sodium hydride (60% in oil, 220 mg, 5.5 mmol) in NMP (5 mL) and allowed to stir for 20 min at rt. At this time, the product from Step 1 (900 mg, 5.0 mmol) was added and the mixture was heated to 120° C. overnight The reaction mixture was cooled, poured into H₂O and extracted with EtOAc. The organic extracts were washed with brine, dried (MgSO₄), filtered and concentrated. The residue was chromatographed (0-5% EtOAc in hex) to give the title compound. MS (ES+) m/e 259 [M+H]⁺.

### Step 3. 4-[1-Ethyl-6-phenyloxy)-1H-benzimidazol-2-yl]-furazan-3-ylamine

The title compound was obtained from the product of Step 2 by the general method of Example 27, Steps 3-5. MS (ES+) m/e 322 [M+H]⁺.

Pharmcy Examples

Tablets

| | | |
|---|---|---|
| a) | Compound of the invention | 50.0 mg |
| | Lactose | 70.0 mg |
| | Microcrystalline Cellulose | 70.0 mg |
| | Cross-linked polyvinylpyrrolidone | 8.0 mg |
| | Magnesium Stearate | 2.0 mg |
| | Compression weight | 200.0 mg |

The compound of the invention, microcrystalline cellulose, lactose and cross-linked polyvinylpyrrolidone are sieved through a 500 micron sieve and blended in a suitable mixer. The magnesium stearate is sieved through a 250 micron sieve and blended with the active blend. The blend is compressed into tablets using suitable punches.

| | | |
|---|---|---|
| b) | Compound of the invention | 50.0 mg |
| | Lactose | 120.0 mg |
| | Pregelatinised Starch | 20.0 mg |
| | Cross-linked polyvinylpyrrolidone | 8.0 mg |
| | Magnesium Stearate | 2.0 mg |
| | Compression weight | 200.0 mg |

The compound of the invention, lactose and pregelatinised starch are blended together and granulated with water. The wet mass is dried and milled. The magnesium stearate and cross-linked polyvinylpyrrolidone are screened through a 250 micron sieve and blended with the granule. The resultant blend is compressed using suitable tablet punches.

Capsules

| | | |
|---|---|---|
| a) | Compound of the invention | 50.0 mg |
| | Lactose | 148.0 mg |
| | Magnesium Stearate | 2.0 mg |
| | Fill weight | 200.0 mg |

The compound of the invention and pregelatinised starch are screened through a 500 micron mesh sieve, blended together and lubricated with magnesium stearate, (meshed through a 250 micron sieve). The blend is filled into hard gelatine capsules of a suitable size.

| | | |
|---|---|---|
| b) | Compound of the invention | 50.0 mg |
| | Lactose | 132.0 mg |
| | Polyvinylpyrrolidone | 8.0 mg |
| | Cross-linked polyvinylpyrrolidone | 8.0 mg |
| | Magnesium Stearate | 2.0 mg |
| | Fill weight | 200.0 mg |

The compound of the invention and lactose are blended together and granulated with a solution of polyvinylpyrrolidone. The wet mass is dried and milled. The magnesium stearate and cross-linked polyvinylpyrrolidone are screened through a 250 micron sieve and blended with the granules. The resultant blend is filled into hard gelatine capsules of a suitable size.

Injection Formulation

| | % w/v |
|---|---|
| Compound of the invention | 0.10 |
| Water for injections B.P. to | 100.00 |

Sodium chloride may be added to adjust the tonicity of the solution and the pH may be adjusted to that of maximum stability and/or to facilitate solution of the compound of the invention using dilute acid or alkali or by the addition of suitable buffer salts. Solubilisers, such as cosolvents, may also be added to facilitate solution of the compound of the invention. Antioxidants and metal chelating salts may also be included. The solution is clarified, made up to final volume with water and the pH remeasured and adjusted if necessary, to provide 1 mg/ml of the compound of formula (I).

The solution may be packaged for injection, for example by filling and sealing in ampoules, vials or syringes. The ampoules, vials or syringes may be aseptically filled (e.g. the solution may be sterilised by filtration and filled into sterile ampoules under aseptic conditions) and/or terminally sterilised (e.g. by heating in an autoclave using one of the acceptable cycles). The solution may be packed under an inert atmosphere of nitrogen.

Preferably the solution is filled into ampoules, sealed by fusion of the glass and terminally sterilised.

Further sterile formulations are prepared in a similar manner containing 0.05, 0.20 and 0.5% w/v of the compound of the invention, so as to provide respectively 0.5, 2 and 5 mg/ml of the compound of the invention.

Biological Activity

Rho-Kinase Activity

Using the test procedure described in the specification the compounds of the examples where found to have a pIC50 in the range 8.5 to 5.1.

Msk-1 Activity

The compounds of the examples have a pIC50 value in the range of 5.0 to 7.7

1. A method of treating an Msk-1 and/or ROCK(1 and/or 2) mediated disease or condition in a mammal comprising administration of an effective amount of a compounds of the general formula (I) 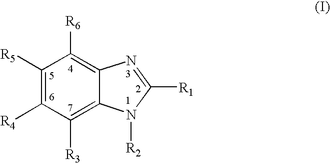
and physiologically acceptable salts thereof wherein,
R₁ is a 5, or 6 membered heterocyclic group selected from group a, b, c or d 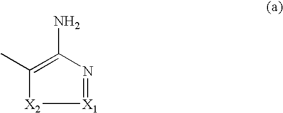
wherein X₁ is a group selected from N or CR₇ and X₂ is a group selected from O, S or NR₈; 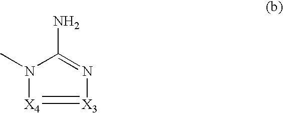
wherein X₃ and X₄ which may be the same or different is a group selected from N or CR₇; 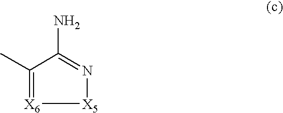
wherein X₅ is a group selected from O, S or NR₈ and X₆ is N or CR₇; 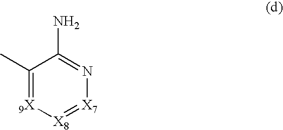
wherein X₇, X₈ and X₉ may be the same or different and selected from a group N or CR₇ R₂ and R₈ independently represents hydrogen, hydroxy, aryl, heteroaryl, C₃₋₇cycloalkyl, heterocyclyl, a group YR₉, N═R₁₀, CONR₁₁R₁₂, COCH₂NR₁₁R₁₂,NR₁₁COR₁₃, SO₂NR₁₁R₁₂ or C₁₋₆alkyl [optionally substituted by a group selected from optionally substituted phenyl, C₃₋₇cycloalkyl, heteroaryl, heterocyclyl, acylamino, NH₂, R₁₆NH, R₁₆R₁₇N, SO₂NR₁₁R₁₂, CONR₁₁R₁₂, NHCOR₁₃, OalkNR₁₆R₁₇, SalkNR₁₆R₁₇ or NR₁₄SO₂R₁₅ group]; or R₂ and R₃ together form a C₂₋₄ alkylene chain.
R₃, R₄, R₅, R₆ and R₇ independently represent a group selected from hydrogen, halogen, hydroxy, R₁₆O, R₁₆S(O)ₙ, NH₂, R₁₆NH, R₁₆R₁₇N, nitro, formyl, C₁₋₄alkanoyl, alkenyl (optionally substituted by optionally substituted phenyl, heterocyclyl, or heteoaryl), carboxy, optionally substituted phenyl, heteroaryl, cycloalkyl, cycloalkylalkyl, aryloxy, heteroaryloxy, heterocyclyl, CONR₁₁R₁₂, NR₁₁COR₁₃, SO₂NR₁₁R₁₂, NR₁₄SO₂R₁₅ or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, acylamino, hydroxy, CONR₁₁R₁₂, NR₁₁COR₁₃, SO₂NR₁₁R₁₂, NR₁₄SO₂R₁₅, OalkNR₁₆R₁₇, or SalkNR₁₆R₁₇ group];
Y represents O, NH,NR₉ or S(O)ₙ;
R₉ represents aryl, heteroaryl, cycloalkyl, heterocyclyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, acylamino, hydroxy, CONR₁₁R₁₂, NR₁₁COR₁₃, SO₂NR₁₁R₁₂, NR₁₄SO₂R₁₅, OalkNR₁₆R₁₇, or SalkNR₁₆R₁₇ group];
R₁₀ represents an alkylidene group which may be substituted by an aryl, heteroaryl, heterocyclyl or cycloalkyl group or R₁₀ represents a cycloalkylidene or heterocycloalkylidene group.
R₁₁ and R₁₂ independently represent hydrogen, aryl, heteroaryl, cycloalkyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, or acylamino group] or R₁₁ and R₁₂ together with the nitrogen atom to which they are attached form a 4-7 heterocyclic ring which may be saturated or unsaturated and optionally contains another heteroatom selected from O,N or S(O)ₙ;
R₁₃ and R₁₅ independently represent, aryl, heteroaryl, heterocyclyl, cycloalkyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, or acylamino group] or the group NR₁₁R₁₂ wherein R₁₁ and R₁₂ have the meanings defined above;
R₁₄ represents hydrogen, aryl, heteroaryl, heterocyclyl, cycloalkyl or C₁₋₆alkyl [optionally substituted by a group selected from optionally substituted phenyl, C₃₋₇cycloalkyl, heteroaryl, heterocyclyl, NH₂, R₁₆NH, R₁₆R₁₇N, or acylamino group];
R₁₆ and R₁₇ independently represent a group selected from C₁₋₆ alkyl, C₃₋₇cycloalkyl, C₃₋₇cycloalkylalkyl, aryl, aralkyl, heteroaryl, heteroarylalkyl, heterocyclyl or heterocyclylalkyl;
Alk is a C₂₋₄ straight or branched alkylene chain
n is zero, 1 or 2.

2. A method as claimed in claim 1 wherein R₁ is the group (c) in which X₅ is oxygen and X₆ is nitrogen.

3. A method as claimed in claim 1 or claim 2 wherein R₂ represents hydrogen, C₁₋₆alkyl alkenyl, alkynyl, C₃₋₇ cycloalkyl, C₃₋C₃₋₇cycloalkylmethyl, phenyl or phenyl substituted by (halogen or aminomethyl), heteroaryl, alkyl substituted by (amino, R₁₆NH or R₁₆R₁₇N), 4-7 membered heterocyclyl group, alkyl substituted by a 4-7 membered heterocyclyl group, a 6 membered heteroaryl group fused to a partially saturated carbocyclic ring, alkyl substituted by optionally substituted phenyl, alkyl substituted by alkenyloxy, or alkyl substituted by trifluoromethyl.

4. A method as claimed in any of claims 1 to 3 wherein R₂ is a group selected from hydrogen, methyl, ethyl, propyl, isopropyl, butyl, allyl, propargyl, cyclopropyl, cyclohexyl, cyclopropylmethyl, cyclobutylmethyl, phenyl or phenyl (substituted by chlorine), pyridyl, 2-aminoethyl, 2-dimethylaminoethyl, 2-amino-1-methylethyl, 2-dimethylamino-1-methylethyl, 4-piperidinyl, t-butyloxycarbonyl-piperidinyl, and 1-ethylpyrrolidin-2-yl-methyl, 3-(4-methyl piperazinyl-1-yl)propyl, 1,2,3,4-tetrahydroisoquinolin-7-yl and the t-butyloxycarbonyl derivative thereof, 1-phenylethyl, 2-vinyloxyethyl, or 2,2,2-trifluoroethyl.

5. A method as claimed in any of claims 1 to 4 wherein R₃ is hydrogen or R₃ and R₂ together represent propylene chain.

6. A method as claimed in any of claims 1 to 5 wherein R₄ is a group selected from hydrogen, chlorine, fluorine, dimethylamino, phenoxy or hydroxy.

7. A method as claimed in any of claims 1 to 6 wherein R₅ is a group selected from hydrogen, methyl, bromine, chlorine, ethoxy, formyl, acetyl, hydroxymethyl or hydroxy.

8. A method as claimed in any of claims 1 to 7 wherein R₈ is a group selected from hydrogen, ethylamino or nitro.

9. A method as claimed in any of claims 1 to 8 wherein the compound of formula (1) is a compound selected from any of examples 1 to 52.

10. A pharmaceutical formulation comprising a compound of formula (I) as defined in claim 1 or a pharmaceutically acceptable salt thereof together with one or more pharmaceutically acceptable carriers or diluents.

11. The use of a compound of formula (I) as defined in claim 1 or a physiologically acceptable salt thereof for the manufacture of a medicament for inhibiting the effects of the kinases Msk-1 and/or ROCK(1 and 2).

12. A compound selected from any of examples 1 to 40 and 48 to 52

13. A process for preparing compounds of formula (I) which comprises:—
a) A process for preparing compounds of formula (I) wherein R₁ is a group (a), (c) and (d) may be prepared by reacting the diamine (II) 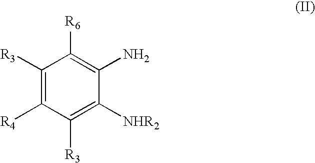
wherein R₂, R₃, R₄, R₅ and R₆ have the meanings defined in (I) with the appropriate compound of formula (III), (IV) or (V) 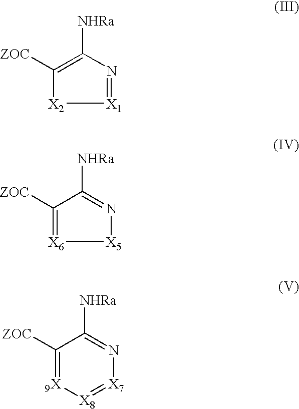
wherein Z is hydrogen, halogen e.g. Cl, Br or I, hydroxy or C₁₋₄alkoxy, Ra is hydrogen or a nitrogen protecting group such as an alkoxycarbonyloxy or benzyloxycarbonyloxy group and each of X₁, X₂, X₅, X₆, X₇, X₈ and X₉ have the meanings as defined in formula (I) or is a group available thereto, followed when required by removal of the nitrogen protecting group Ra using conventional methods.
b) A process for preparing compounds of formula (I) wherein R₁ is the group (b) by reducing of the corresponding nitro derivative (VI) 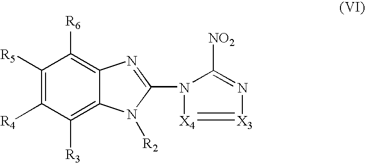
wherein R₂, R₃, R₄, R₅, R₆, X₃ and X₄ have the meanings defined in formula (I).
c) a process for preparing compounds of formula (I) wherein R₁ is the group (c) and X₅ is oxygen and X₆ is nitrogen by reacting the nitrile (VII) 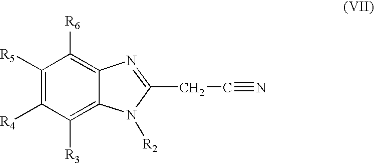
wherein R₂, R₃, R₄ R₅ and R₆ have the meanings defined in formula (I) with hydrochloric acid and sodium nitrite in a solvent and treatment of the product thus formed with a base and hydroxylamine.
d) A process for preparing compounds of formula (I) wherein R₁ is the group (c) and X₅ is oxygen and X₆ is nitrogen by reacting the diamine (II) with 5-methyl-[1,4,5]oxadiazolo[3,4-d]pyrimidin-7-ol in glacial acetic acid.
e) a process for preparing compounds of formula (I) wherein R₁ represent the group (c) wherein X₅ is NH and X₆ is CH by reacting compound (VIII) 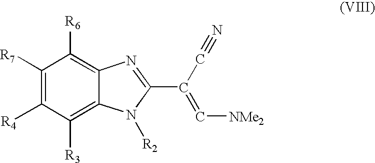
wherein R₂, R₃, R₄ R₅ and R₁ have the meanings defined in formula (I) with hydrazine.
